(19) United States
(12) Patent Application Publication
(10) Pub. No.: US 2021/0318335 A1
(43) Pub. Date: Oct. 14, 2021

(54) BIOMARKERS, COMPOSITIONS, AND METHODS FOR DIAGNOSING AND TREATING SUBJECTS EXPOSED TO PROTEIN/HEPARIN COMPLEXES

(71) Applicant: DUKE UNIVERSITY, Durham, NC (US)

(72) Inventors: Sanjay Khandelwal, Durham, NC (US); Gowthami Arepally, Durham, NC (US)

(73) Assignee: DUKE UNIVERSITY, Durham, NC (US)

(21) Appl. No.: 17/271,841

(22) PCT Filed: Aug. 28, 2019
(86) PCT No.: PCT/US2019/048669

§ 371 Date: Feb. 26, 2021

Related U.S. Application Data
(60) Provisional application No. 62/723,720, filed on Aug. 28, 2018.

(51) Int. Cl. G01N 33/68; C07K 16/18; A61K 31/713

(52) U.S. Cl. CPC G01N 33/6854; C07K 16/18; G01N 2800/24; G01N 2800/50; A61K 31/713

(57) ABSTRACT

The present disclosure provides methods of determining the presence of the risk of developing complement activation by protein/heparin binding complexes in a subject, methods of determining the presence of complement activation by protein/heparin binding complexes in a subject, and methods of treating diseases including heparin-induced thrombocytopenia (HIT) in a subject by administering a compound capable of blocking the classical pathway of complement activation.

### CROSS-REFERENCE TO RELATED APPLICATIONS

This application claims priority to U.S. Provisional Patent Application No. 62/723,720, filed Aug. 28, 2018, the disclosure of which is hereby incorporated by cross-reference in its entirety.

### FEDERAL FUNDING LEGEND

This invention was made with Government support under Federal Grant No. P01-HL110860 and R01 HL136512 awarded by the NIH/NHLBI. The Federal Government has certain rights to this invention.

### BACKGROUND

### Field

The present disclosure provides methods of determining the presence of the risk of developing complement activation by protein/heparin binding complexes in a subject, methods of determining the presence of complement activation by protein/heparin binding complexes in a subject, and methods of treating diseases including heparin-induced thrombocytopenia (HIT) in a subject by administering a compound capable of blocking the classical pathway of complement activation.

### Description of Related Art

Autoantibodies to platelet factor 4 (PF4)/heparin develop in 25-50% of patients exposed to heparin during cardiopulmonary bypass (CPB) and cause heparin-induced thrombocytopenia (HIT) in a subset of patients. This striking propensity for antibody formation in clinic settings such as cardiac surgery remains unexplained. The high incidence of heparin sensitization is difficult to reconcile with known mechanisms of MHC-restricted antigen presentation. One potential explanation derives from recent findings showing robust complement activating activities of ultra-large complexes of PF4/heparin (ULCs). PF4/heparin ULCs activate complement in plasma in a heparin-dependent manner both in vitro and in patients receiving heparin therapy. Complement activation by PF4/heparin ULCs elicits binding of C3 fragments to antigen and facilitates antigen deposition on circulating B cells via the complement receptor 2/CD21. As binding of complement coated antigen to CD21 potentiates its immunogenicity 1000-10,000 fold, complement activation and subsequent binding of PF4/heparin to B cells can represent early, sensitizing events in recipients of heparin.

How PF4/heparin complexes activate complement is unknown. Complement can be activated by the classical, alternative or lectin pathways individually or in combination. Several lines of evidence implicate involvement of the classical pathway in HIT. Studies performed in the 1960's and 1970's showed that mixtures of polycations and polyanions, such as protamine (PRT)/heparin and lysozyme/DNA, activate the classical pathway of complement, possibly through a non-immune mechanism. More recently, anti-PF4/heparin reactive immunoglobulin (Ig) M has been identified in healthy donor blood.

There is a need for new therapeutic agents and methods to assess and determine the risk of developing complement activation by protein/heparin binding complexes in a subject, as well as a need to prevent antibody generation in diseases such HIT. In the studies described herein, it was shown that non-immune, naturally occurring IgM mediates complement activation by PF4/heparin complexes and promotes antigen deposition on B cells. Additionally, the studies described herein suggest that pre-exposure levels of plasma IgM may constitute a stable biomarker for the risk of sensitization and possible development of HIT.

### BRIEF SUMMARY OF THE DISCLOSURE

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

An embodiment provides a method of determining the presence of the risk of developing complement activation by protein/heparin binding complexes in a subject. The method can comprise obtaining a biological sample from the subject, determining the presence of plasma IgM in the biological sample, and, if the plasma IgM is determined in an amount greater than a control, administering to the subject a therapeutically effective amount of a compound capable of blocking the classical pathway of complement activation such that the complement activation by protein/heparin complexes is blocked in the subject.

The protein of the protein/heparin binding complex can comprise platelet factor 4 (PF4). The compound can be a complement inhibitor. The complement inhibitor can be an antibody, antisense RNA, cDNA, small molecule, fusion protein, peptide, oligonucleotide. In some embodiments, the the complement inhibitor is eculizumab, C1-INH, anti-C1q antibodies, anti-C1s antibodies, or compstatin, anti-CD21 inhibitors.

In some embodiments, the concentration of plasma IgM in the biological sample that is at about 200 μg/mL to about 3000 μg/mL can indicate an increased likelihood of developing complement activation by protein/heparin binding complexes in the subject.

In some embodiments, the compound capable of blocking the classical pathway of complement activation can be administered in a pharmaceutically acceptable composition.

In some embodiments, the pharmaceutically acceptable composition can comprise a pharmaceutically acceptable carrier. In other embodiments, the pharmaceutically acceptable carrier can be a gene, polypeptide, antibody, liposome, polysaccharide, polylactic acid, polyglycolic acid, or an inactive virus particle.

In some embodiments, the compound capable of blocking the classical pathway of complement activation can be administered intravenously, intraperitonealy, intramuscularly, subcutaneously, or transdermaly.

The biological sample can be tissues, cells, biopsies, blood, lymph, serum, plasma, urine, saliva, mucus, or tears. In some embodiments, the biological sample is a tumor biopsy.

Yet another embodiment provides method of determining the presence of complement activation by protein/heparin binding complexes in a subject. The method can comprise obtaining a biological sample from the subject, determining the presence of plasma IgM in the biological sample, and, if the plasma IgM is determined in an amount greater than the control, administering to the subject a therapeutically effective amount of a compound capable of blocking the classical pathway of complement activation such that complement activation by protein/heparin complexes is blocked in the subject.

The protein of the protein/heparin binding complex can comprise platelet factor 4 (PF4). The compound can be a complement inhibitor. The complement inhibitor can be an antibody, antisense RNA, cDNA, small molecule, fusion protein, peptide, oligonucleotide. In some embodiments, the complement inhibitor is eculizumab, C1-INH, anti-C1q antibodies, anti-C1s antibodies, compstatin, or anti-CD21 inhibitors.

In some embodiments, the amount of plasma IgM in a biological sample can be about 200 μg/mL to about 3000 μg/mL.

In some embodiments, the compound capable of blocking the classical pathway of complement activation can be administered in a pharmaceutically acceptable composition.

In some embodiments, the pharmaceutically acceptable composition can comprise a pharmaceutically acceptable carrier. In other embodiments, the pharmaceutically acceptable carrier can be a gene, polypeptide, antibody, liposome, polysaccharide, polylactic acid, polyglycolic acid, or an inactive virus particle.

In some embodiments, the compound capable of blocking the classical pathway of complement activation can be administered intravenously, intraperitonealy, intramuscularly, subcutaneously, or transdermaly.

The biological sample can be tissues, cells, biopsies, blood, lymph, serum, plasma, urine, saliva, mucus, or tears. In some embodiments, the biological sample is a tumor biopsy.

Yet another embodiment provides a method of treating a disease characterized by the onset of antibody generation or preventing the onset of a disease characterized by the onset of antibody generation in a subject. The method can comprise administering to the subject a therapeutically effective amount of a compound capable of blocking the classical pathway of complement activation such that antibody generation is blocked in the subject and the onset of disease is prevented or the disease is treated. The disease can comprise heparin-induced thrombocytopenia (HIT).

The compound can be a complement inhibitor. The complement inhibitor can be an antibody, antisense RNA, cDNA, small molecule, fusion protein, peptide, oligonucleotide. In some embodiments, the the complement inhibitor is eculizumab, C1-INH, anti-C1q antibodies, anti-C1s antibodies, compstatin, or anti-CD21 inhibitors.

In some embodiments, the compound capable of blocking the classical pathway of complement activation can be administered in a pharmaceutically acceptable composition.

In some embodiments, the pharmaceutically acceptable composition can comprise a pharmaceutically acceptable carrier. In other embodiments, the pharmaceutically acceptable carrier can be a gene, polypeptide, antibody, liposome, polysaccharide, polylactic acid, polyglycolic acid, or an inactive virus particle.

In some embodiments, the compound capable of blocking the classical pathway of complement activation can be administered intravenously, intraperitonealy, intramuscularly, subcutaneously, or transdermaly.

Yet another embodiment provides a method of treating heparin-induced thrombocytopenia (HIT) in a subject, the method comprising administering to the subject a therapeutically effective amount of a compound capable of blocking the classical pathway of complement activation such that the HIT is treated in the subject.

In some embodiments, the compound capable of blocking the classical pathway of complement activation can be administered in a pharmaceutically acceptable composition.

In some embodiments, the pharmaceutically acceptable composition can comprise a pharmaceutically acceptable carrier. In other embodiments, the pharmaceutically acceptable carrier can be a gene, polypeptide, antibody, liposome, polysaccharide, polylactic acid, polyglycolic acid, or an inactive virus particle.

In some embodiments, the compound capable of blocking the classical pathway of complement activation can be administered intravenously, intraperitonealy, intramuscularly, subcutaneously, or transdermaly.

Additional features and advantages are described herein, and will be apparent from the following Detailed Description, Drawings, and the claims.

### BRIEF DESCRIPTION OF THE DRAWINGS

The foregoing aspects and other features of the disclosure are explained in the following description, taken in connection with the accompanying drawings, wherein:

FIG. 1 is a schematic showing the mechanism of complement activation by PF4/heparin complexes. Step A: Heparin displaces PF4 to form ULCs. Step B: Polyreactive natural IgM from plasma binds to ULCs, changes conformation and binds C1q to activate the classical pathway of complement activation. Step C: IgM and complement-coated antigen binds to B cells via complement receptor 2.

FIG. 2A-2C show that complement activation in response to PF4/heparin complexes among healthy donors defines a donor “phenotype.” FIG. 2A is a graph showing that an ELISA based antigen capture assay detects C′ activation by PF4/heparin. FIG. 2B is a graph showing the anti-C3c absorbance of donor plasma incubated with different antigens. Each symbol represents an individual donor. Results are shown from a representative experiment performed a minimum of three times with multiple donors in each experiment. FIG. 3C is a graph showing % PF4/heparin complement activation relative to donor 1 on different days. ** p<0.0001. Complement activation by PF4/heparin of donors (1, 2, 3, & 4) was determined over a period of 626 days (˜1.7 years) and normalized to donor 1 who was studied at all time points.

FIG. 3A-3B show complement activation and PF4/heparin binding to B cells in healthy donors. FIG. 3A is a histogram showing the binding of anti-PF4/heparin (KKO) on the B cells for the three donors. From left to right, the peaks represent: isotype control staining (filled peak), Low C′ (donor 4), Intermediate C′ (donor 1), and High C′ (donor 2). FIG. 3B is a histogram showing the binding of C3c on the B cells for the three donors. From left to right, the peaks represent: isotype control staining (filled peak), Low C′ (donor 4), intermediate C′ (donor 1), and High C′ (donor 2).

FIG. 4A-4F show complement activation by PF4/heparin correlates with plasma/serum IgM levels. FIG. 4A is a graph showing the PF4/heparin induced C′ activation by different donors (determined by ELISA based antigen capture assay) and their plasma IgM levels (quantified by proteomic analysis). For each point on the x-axis, the left bar represents C3c and the right bar represents IgM. FIG. 4B is a graph showing proteomic analysis of plasmas with a high, intermediate or low complement response phenotype shows strong correlation with plasma IgM. The graph shows IgM protein intensity determined by proteomic analysis (x-axis) and complement activation response to PF4/heparin as measured by the antigen-C3c capture ELISA assay (y-axis). FIG. 4C is a graph showing serum immunoglobulin levels of IgM from 29 healthy donors were measured in the clinical laboratory and correlated with an individual's complement activation response to PF4/heparin as measured by the antigen-C3c capture ELISA assay. FIG. 4D is a graph showing serum immunoglobulin levels of IgG from 29 healthy donors were measured in the clinical laboratory and correlated with an individual's complement activation response to PF4/heparin as measured by the antigen-C3c capture ELISA assay. FIG. 4E is a graph showing serum immunoglobulin levels of IgA from 29 healthy donors were measured in the clinical laboratory and correlated with an individual's complement activation response to PF4/heparin as measured by the antigen-C3c capture ELISA assay. The graphs show correlation of complement activation (y-axis) as a function of immunoglobulin levels (x-axis). Each symbol in the graph represents an individual donor. Complement activation values were normalized to an intermediate donor studied in parallel. FIG. 4F is a graph showing binding of plasma IgM from high C′ phenotype donors (n=3) to different antigens as determined by ELISA on a microtiter plate coated with, from left to right of the bars for each donor on the x-axis, PF4 alone (10 μg/mL) or PF4 (10 μg/mL)+Heparin (0.4 U/mL) or Protamine sulfate (PRT; 31 μg/mL)+heparin (4 U/mL).

FIG. 5A-5C show plasma IgM mediates complement activation by PF4/heparin complexes. FIG. 5A is a graph showing complement activation (y-axis) as a function of added immunoglobulin concentration. Commercial IgM (0-1000 μg/mL; filled symbols) or IgG (0-5000 μg/mL; open symbols) or monoclonal myeloma IgM (0-1000 μg/mL; hatched symbols) was added to the plasmas of two donors with a “low” complement response type (circle/square) and complement activation by PF4/heparin was measured by the antigen-C3c capture ELISA assay. FIG. 5B is a graph showing complement activation (y-axis) in control or IgM depleted plasma (x-axis). Plasma with an “intermediate” donor phenotype was incubated with anti-IgM or control beads, followed by addition of buffer, PF4/heparin or PF4/heparin+400 μg/mL IgM and complement, and activation was measured by the antigen-C3c capture ELISA assay. FIG. 5C is a graph showing complement activation response at varying IgM concentrations (y-axis) as a function of PF4/heparin concentrations (x-axis). Plasma with a “low” donor phenotype was incubated with varying antigen concentrations (PF4; 0-25 μg/mL+Heparin; 0-0.25 U/mL) and IgM (0-800 μg/mL), and complement activation was measured by the antigen-C3c capture ELISA assay. *p<0.005 and **p<0.0001. Results are shown from a representative experiment involving three donors tested on three different occasions.

FIG. 6A-6D show polyreactive IgM mediates complement activation by PF4/heparin. FIG. 6A is a graph showing the binding (y-axis) of various concentrations of commercial IgM (1.25 μg/mL−80.0 μg/mL) to different antigens. Antigen-specificity of commercial IgM was determined using microtiter plates coated with various antigens. FIG. 6B is a graph showing antigen-specificity of IgM in the plasma of high complement response donor, which was determined using microtiter plates coated with various antigens. Graph shows the binding IgM (y-axis) to various antigens at different plasma dilutions. FIG. 6C is a graph showing antigen-specificity of IgM in the plasma of intermediate complement response donor, which was determined using microtiter plates coated with various antigens. Graph shows the binding of IgM (y-axis) to various antigens at different plasma dilutions. FIG. 6D is a graph showing antigen-specificity of IgM in the plasma of low complement response donor, which was determined using microtiter plates coated with various antigens. Graph shows the binding of IgM (y-axis) to various antigens at different plasma dilutions.

FIG. 7A-7B show polyreactivity of PF4/heparin binding IgM. FIG. 7A is a graph showing the binding of IgM (y-axis) to various antigens. PF4/heparin binding IgM were isolated from the pooled healthy donor IgM by using a PF4/heparin column. Polyreactivity of these isolated PF4/heparin binding IgM (2 μg/mL) was determined by using microtiter plates coated with various antigens. FIG. 7B is a graph showing the binding of unfractionated IgM (prior to separation by PF4/heparin column; y-axis) to various antigens. Antigen binding specificity of unfractionated IgM (pooled healthy donor IgM; 20 μg/mL) was determined by using microtiter plates coated with various antigens.

FIG. 8A-8D show that polyreactive IgM mediates complement activation by PRT/heparin complexes. FIG. 8A is a graph showing complement activation (y-axis) in control or IgM depleted plasma (x-axis) ** p<0.0001. Plasma of an “intermediate” donor phenotype was treated with anti IgM or control beads, followed by addition of buffer, PRT (125 μg/mL)/heparin (6 U/mL) or PRT/heparin+400 μg/mL IgM and complement activation was measured by antigen-C3c capture ELISA assay on a mouse anti PRT/heparin antibody (ADA) coated plate. FIG. 8B is a graph showing complement activation (y-axis) as a function of an added polyreactive, monoclonal IgM antibody. Results are shown from a representative experiment involving three donors tested on three different occasions. ** p<0.0001, relative to no polyreactive IgM added. Complement activation by polyreactive monoclonal IgM, 2E4, in the plasma of two donors (“low” complement activation phenotype; circle/square) in response to buffer (open symbols), PF4 alone (hatched symbols), heparin alone (half-filled symbols) and PF4/heparin (filled symbols) as measured by the antigen-C3c capture ELISA assay. FIG. 8C is a graph showing complement activation (y-axis) in different incubation conditions with and without added polyreactive monoclonal IgM (10 μg/mL, 2E4). ** p<0.0001, relative to no polyreactive IgM added. Complement activation by polyreactive monoclonal IgM, 2E4, in the plasma of an “intermediate” donor phenotype in response to buffer and PRT (125 μg/mL)±heparin (6 U/mL) was measured by antigen-C3c capture ELISA assay. FIG. 8D are histograms showing the representative results from two different experiments with two different cord blood samples. Whole blood from the cord blood of a baby was incubated with buffer or PF4±heparin and binding of PF4/heparin (KKO) or C3c to the B cells was determined by flow cytometry.

FIG. 9A-9G show PF4/heparin activate complement by classical pathway. FIG. 9A is a graph showing complement activation in different incubation conditions. Plasma from a healthy donor was incubated with EDTA (10 mM) or EGTA (10 mM)±MgCl₂ (10 mM) or with buffer before incubating with PF4/heparin and complement activation was measured by the antigen-C3c capture ELISA assay. ***p<0.0001. Results are shown from a representative experiment involving three donors tested on three different occasions. FIG. 9B is a graph showing the complement activation in different incubation conditions. Plasma from a healthy donor was incubated with or without C1-inhibitor (10 and 20 IU/mL) before incubating with PF4/heparin and complement activation by PF4/heparin was determined by antigen-C3c capture ELISA assay. ***p<0.0001. FIG. 9C is a histogram showing the binding of anti-PF4/heparin (KKO) to B cells in various incubation conditions. The overlapping peaks represent buffer control (striped lines), followed by PF4, PF4/heparin+EDTA, PF4/heparin+EGTA+MgCl2, and PF4/heparin+EGTA. Peak 1 represents PF4/heparin. FIG. 9D is a histogram showing the binding of anti-C3c to B cells in various incubation conditions. The overlapping peaks represent PF4/heparin+EDTA, PF4/heparin+EGTA, PF4/heparin+EGTA+MgCl2, and buffer control (striped lines), and PF4. Peak 1 represents PF4/heparin. FIG. 9E is a graph showing complement activation in presence of various antibodies. Plasma from a healthy donor was incubated with various concentration of anti-C1q antibody, anti-MBL antibody or control antibody (0-100 μg/mL before adding PF4/heparin and complement activation by PF4/heparin was determined by the antigen-C3c capture ELISA assay. * p<0.05, ** p<0.001, *** p<0.0001, compared to with no antibody added condition. Results are shown from a representative experiment involving three donors tested on three different occasions. FIG. 9F is a histogram showing the binding of anti-PF4/heparin to B cells in various incubation conditions. The peaks represent the buffer control (striped line), anti-C1q+PF4/heparin (peak 1), anti-MBL+PF4/heparin (peak 2), PF4/heparin (peak 3), and MS IgG 1+PF4/heparin (peak 4). FIG. 9G is a histogram showing the binding of anti-C3c to B cells in various incubation conditions. The peaks represent the buffer control (striped line), anti-C1q+PF4/heparin (peak 1), anti-MBL+PF4/heparin (peak 2), PF4/heparin (peak 3), and MS IgG 1+PF4/heparin (peak 4).

FIG. 10A-10D shows plasma IgM co-localizes with PF4/heparin and C3 fragments on B cells in healthy donors and patients on heparin therapy. FIG. 10A is a histogram overlay showing binding of PF4/heparin (KKO)/C3c/IgM to B cells is shown with and without PF4±heparin in normal and excess heparin wash conditions. Results are shown from a representative experiment involving three donors tested on three different occasions. FIG. 10B is a graph showing mean fluorescent intensity of PF4/heparin (KKO)/C3c/IgM to B cells is shown with and without PF4±heparin in normal and excess heparin wash conditions. Peak 1 represents PF4/heparin. Results are shown from a representative experiment involving three donors tested on three different occasions. FIG. 10C is a histogram overlay showing binding of anti-PF4/heparin (KKO)/C3c/IgM to B cells is shown pre- and post-heparin exposure in the patient. Results are shown from one representative patient out of two patients studied. FIG. 10D is a graph showing the mean fluorescence intensity of binding of anti-PF4/heparin (KKO)/C3c/IgM to B cells is shown pre- and post-heparin exposure in the patient. Results are shown from one representative patient out of two patients studied.

### DETAILED DESCRIPTION OF THE DISCLOSURE

For the purposes of promoting an understanding of the principles of the present disclosure, reference will now be made to embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e., at least one) of the grammatical object of the article. By way of example, “an element” means at least one element and can include more than one element.

As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

As used herein, “about” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of and “consisting of those certain elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially or” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B, and C, it is specifically intended that any of A, B, or C, or a combination thereof, can be omitted and disclaimed singularly or in any combination.

“Optional” or “optionally” means that the subsequently described circumstance may or may not occur, so that the description includes instances where the circumstance occurs and instances where it does not.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

Unless otherwise defined, all terms (including technical and scientific terms) used herein have the same meaning as commonly understood by one having ordinary skill in the art to which this disclosure belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

It will be understood that a number of aspects and embodiments are disclosed. Each of these has an individual benefit and each can also be used in conjunction with one or more, or in some cases all, of the other disclosed aspects and embodiments, whether specifically delineated or not. Accordingly, for the sake of clarity, this description will refrain from repeating every possible combination of the individual aspects and embodiments in an unnecessary fashion. Nevertheless, the specification and claims should be read with the understanding that such combinations are implicitly disclosed, and are entirely within the scope of the invention and the claims, unless otherwise specified.

The present disclosure is based, in part, on the findings relating to the process of how platelet factor 4 (PF4)/heparin complexes activate complement in plasma and bind to B cells. In particular, it was found that (1) plasma IgM levels correlate with functional complement responses to PF4/heparin; and (2) polyreactive IgM binds PF4/heparin, thereby triggering activation of the classical complement pathway and promoting antigen and complement deposition on B cells.

The studies described herein also show that plasma IgM levels are highly correlated with the degree of complement activation by PF4/heparin complexes. Thus, heparin pre-exposure levels of plasma IgM can be used as a stable biomarker for the risk of anti PF4/heparin antibody generation and subsequent HIT.

Accordingly, one aspect of the present disclosure provides a method of determining the presence of the risk of developing complement activation by protein/heparin binding complexes in a subject, the method comprising: (a) obtaining a biological sample from the subject; (b) determining the presence of plasma IgM in the biological sample; and (c) if the plasma IgM is determined in an amount greater than a control, administering to the subject a therapeutically effective amount of a compound capable of blocking the classical pathway of complement activation such that complement activation by protein/heparin complexes is blocked in the subject.

The “risk of developing complement activation by protein/heparin binding complexes in a subject” refers to the likelihood or chance that the subject will develop protein/heparin binding complexes. In some embodiments, the risk of developing complement activation by protein/heparin binding complexes in a subject can be a 5% likelihood, 10% likelihood, 25% likelihood, 30% likelihood, 35% likelihood, 40% likelihood, 50% likelihood, 55% likelihood, 60% likelihood, 65% likelihood, 70% likelihood, 75% likelihood, 80% likelihood, 85% likelihood, or greater.

In some embodiments, the presence of plasma IgM in the biological sample in an amount greater than a control sample can indicate an increased likelihood of developing complement activation by protein/heparin binding complexes. In some embodiments, the presence of plasma IgM in the biological sample in an amount greater than 5%, 10%, 15%, 20%, 25%, 30%, 35%, 40%, 45%, 50%, 60%, 70%, 80%, 90% or greater than the amount of plasma IgM in a control sample can indicate an increased likelihood of developing complement activation by protein/heparin binding complexes in the subject.

In other embodiments, the presence of plasma IgM in the biological sample in an amount greater than 2 fold, 5 fold, 10 fold, 15 fold, 20 fold, or greater than the amount of plasma IgM in a control sample can indicate an increased likelihood of developing complement activation by protein/heparin binding complexes in the subject.

In some embodiments, the presence of plasma IgM in the biological sample in an amount greater than or equal to 200 μg/mL (e.g., about 250 μg/mL, about 300 μg/mL, about 350 μg/mL, about 400 μg/mL, about 450 μg/mL, or about 500 μg/mL, or about 600 μg/mL, or about 700 μg/mL, or about 800 μg/mL, or about 900 μg/mL, or about 1000 μg/mL, or about 1100 μg/mL, or about 1200 μg/mL, or about 1300 μg/mL, or about 1400 μg/mL, or about 1500 μg/mL, or about 1600 μg/mL, or about 1700 μg/mL, or about 1800 μg/mL, or about 1900 μg/mL, or about 2000 μg/mL, or about 2100 μg/mL, or about 2200 μg/mL, or about 2300 μg/mL, or about 2400 μg/mL, or about 2500 μg/mL, or about 2600 μg/mL, or about 2700 μg/mL, or about 2800 μg/mL, or about 2900 μg/mL, or about 3000 μg/mL, or greater) can indicate an increased likelihood of developing complement activation by protein/heparin binding complexes in the subject. In some embodiments, the presence of plasma IgM in the biological sample in a concentration of about 200 μg/mL to about 3000 μg/mL can indicate an increased likelihood of developing complement activation by protein/heparin binding complexes in the subject.

A person of ordinary skill in the art will understand based on the present disclosure and knowledge in the art how to determine a proper control for the methods described herein. For example, a control can be plasma that has not been exposed to protein/heparin binding complexes. A control can be plasma containing IgM from a healthy subject without prior heparin exposure. A control can also be a buffer solution.

The term “biological sample” as used herein includes, but is not limited to, a sample containing tissues, cells, and/or biological fluids isolated from a subject. Examples of biological samples include, but are not limited to, tissues, cells, biopsies, blood, lymph, serum, plasma, urine, saliva, mucus and tears. In one embodiment, the biological sample is a biopsy (such as a tumor biopsy). A biological sample can be obtained directly from a subject (e.g., by blood or tissue sampling) or from a third party (e.g., received from an intermediary, such as a healthcare provider or lab technician).

The “protein/heparin binding complex” includes a protein that binds to heparin with a high affinity. The protein component of the “protein/heparin binding complex” can be, for example, a cytokine, an extracellular protein, a growth factor, a chemokine, an enzyme, or a lipoprotein. The protein can be an endogenous protein or a synthetic protein. The protein can be any protein that binds to heparin with a high affinity (e.g., PF4, protamine sulfate, antithrombin, fibroblast growth factors, hepatocyte growth factor, interleukin-8, vascular endothelial growth factor, wnt/wingless, or endostatin). The protein can be any protein that is positively charged. In some embodiments, the protein of the protein/heparin binding complex comprises platelet factor 4 (PF4).

Platelet factor 4 (PF4) is a cytokine belonging to the CXC chemokine family that is also referred to as chemokine (C—X—C motif) ligand 4 (CXCL4). PF4 is a 70 amino acid protein that can be released from alpha-granules of activated platelets during platelet aggregation and promotes blood coagulation by moderating the effects of heparin and heparin-like molecules. PF4 has a high binding affinity to heparin. The heparin/PF4 complex is the antigen in heparin-induced thrombocytopenia (HIT). HIT is an autoimmune reaction to the administration of the anticoagulant, heparin. PF4 autoantibodies have also been found in patients with thrombosis and patients with features resembling HIT, but no prior administration of heparin.

The heparin component of the “protein/heparin binding complex” can be a glycosaminoglycan family member that is highly sulfated and negatively charged. Heparin can be naturally occurring in the body or can be administered as a medication. Heparin can act as an anticoagulant (blood thinner). Heparin is also known as unfractionated heparin (UFH). The term heparin can also include derivatives (e.g., enoxaparin, dalteparin, tinzaparin), heparin-like molecules (e.g, sulfated glycosaminoglycans, including heparan sulfate). The term heparin can include other carbohydrate moieties that are negatively charged, such as naturally occurring glycosaminoglycans (e.g., chondroitin sulfate, heparan sulfate, dextran sulfate, or dermatan sulfate), synthetic glycosaminoglycans, RNA/DNA molecules that are negatively charged, polyphosphates, and any negatively charged molecules that can bind positively charged proteins to generate “protein/heparin-like complexes.”

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like.

The term “antibody” herein is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

Immunoglobulins are types of antibodies. There are five immunoglobulin classes (isotypes) of antibody molecules found in serum: IgG, IgM, IgA, IgE and IgD. Immunoglobulin can be distinguished by the type of heavy chain polypeptide they contain. IgG molecules can contain heavy chains known as γ-chains, IgMs can contain μ-chains, IgAs can contain α-chains, IgEs can contain ε-chains, and IgDs can contain δ-chains. The variation in heavy chain polypeptides allows each immunoglobulin class to function in a different type of immune response or during a different stage of the body's defense.

Immunoglobulin M (IgM) can be found in all body fluids and protects against bacterial and viral infections. In mice and humans, IgM can occur either as a membrane-bound monomer on B cells (as a part of B cell receptor, BCR) or as a secreted, pentameric protein in plasma. IgM can be further divided into “natural” or “immune” based on antigen-binding specificities. Natural IgM can be detected in normal quantities in mice grown under antigen/germ-free conditions, can arise from endogenous antigens, can display reactivity to a wide range of self and foreign antigens, and can exhibit germline-encoded variable heavy- and light-chain genes. Immune IgM, on the other hand, represents an antigen-specific immune response to pathogens or external antigens with limited cross-reactivity and presence of highly mutated variable gene regions.

The polyreactivity of natural IgM endows it with diverse sentinel functions in health and disease. In infection, natural IgM can faciliate antigen-specific immunity by binding pathogens, triggering complement activation, and transporting antigen via noncognate B cells to splenic subcompartments. Mice lacking natural IgM exhibit defective antigen trapping of particulate antigen, impaired germinal center formation, increased susceptibility to bacterial and viral pathogens and defective T-cell dependent immunity. These defects mirror the phenotypes seen with deficiencies of classical pathway components and/or the complement receptor CD21, indicating an interconnectedness of pathways involving natural IgM, complement, and CD21.

Immunoglobulin A (IgA) can be found in high concentrations in the mucous membranes, including but not limited to the membranes lining the respiratory passages and gastrointestinal tract. IgA can also be found in saliva, milk, and tears.

Immunoglobulin G (IgG), is the most abundant type of antibody, and can be found in all body fluids. IgG can protect against bacterial and viral infections.

Immunoglobulin E (IgE) is associated with allergic reactions (e.g., when the immune system overreacts to environmental antigens such as dust). IgE can be found in the lungs, skin, and mucous membranes.

Immunoglobulin D (IgD) can exists in small amounts in the blood and can be expressed on the surface of mature B cells.

The term “effective amount” or “therapeutically effective amount” refers to an amount sufficient to effect beneficial or desirable biological and/or clinical results. For example, as used herein a therapeutically effective amount of a compound capable of blocking the classical pathway of complement activation can inhibit, prevent, reduce, or block the complement activation by protein/heparin complexes in a subject.

The “classical pathway of complement activation” refers to one of three pathways that activate the complement system, which is part of the immune system. The classical complement pathway is initiated by antigen-antibody complexes with the antibody isotypes IgG and IgM. The other two pathways that can activate the complement system are the alternative complement pathway and the lectin pathway.

The classical pathway can be triggered by activation of the C1-complex. The C1-complex is composed of one molecule of C1q, two molecules of C1r and 2 molecules of C1s, or C1qr₂s₂. This occurs when C1q binds to IgM or IgG complexed with antigens. A single pentameric IgM can initiate the pathway. This also occurs when C1q binds directly to the surface of the pathogen. Such binding leads to conformational changes in the C1q molecule, which leads to the activation of two C1r molecules. C1r is a serine protease. They then cleave C1s (another serine protease). The C1r₂s₂ component now splits C4 and then C2, producing C4a, C4b, C2a, and C2b (historically, the larger fragment of C2 was called C2a but can now be referred to as C2b). C4b and C2b bind to form the classical pathway C3-convertase (C4b2b complex), which promotes cleavage of C3 into C3a and C3b. C3b later joins with C4b2b to make C5 convertase (C4b2b3b complex).

The “complement system” is a part of the immune system that enhances (complements) the ability of antibodies and phagocytic cells to clear microbes and damaged cells from an organism, promote inflammation, and attack the pathogen's cell membrane. The complement system is part of the innate immune system. The complement system can, also be recruited and brought into action by antibodies generated by the adaptive immune system.

A “compound capable of blocking the classical pathway of complement activation,” or a “complement inhibitor,” as used herein, refers to a molecule that inhibits the activity of a complement molecule that acts in, or has an indirect interaction with, the classical complement pathway. Alternatively, a complement inhibitor refers to a molecule that, when administered to a subject, organism, or cell, results in a phenotype that indicates inhibition of the activity of a complement molecule, and in one embodiment is a molecule that results in a phenotype that indicates specific inhibition of the activity of a specific target complement component molecule.

The term “block” or “blocking” as used herein refers to decreasing or inhibiting the activity of one or more molecules in the classical pathway of complement activation (e.g., C1q, C1r, C1s, C3, C3a, C5a, etc.) such that complement activation is decreased, reduced, inhibited, or prevented.

A complement inhibitor as described herein can be an antibody, antisense RNA, cDNA, small molecule, fusion protein, peptide, oligonucleotide, and the like. Examples of suitable complement inhibitors additionally may include, but are not limited to, for example, eculizumab (Solaris™), anti-C1q antibodies, Soluble Human Complement Receptor Type 1 (sCR1); Vaccinia CCP (Vaccinia complement control protein), soluble decay-accelerating factor (sDAF), soluble membrane cofactor protein (sMCP), a fusion protein comprising sMCP fused to DAF (sMCP-DAF), soluble CD59 (sCD59), a fusion protein comprising DAF fused to CD59 (DAF-CD59) (as taught, for example, in U.S. Patent Publication 2008/0267980), C5a mutants, Anti-C3 antibody, Anti-C5a antibody, Anti-C3a antibody, the C5aR antagonists N MeFKPdChaWdR and F-(OpdChaWR)C5aR, RNA aptamers that inhibit human complement C5 (see, e.g., Biesecker et al., Immunopharmacology, 42(1-3):219-230 (1999)), BCX-1470, FUT-175, K-76, thioester inhibitors, C1-INH (Cetor/Sanquin, BerinertP/CSL Behring, Lev Pharma), Rhucin/rhC1 INH (Pharming Group N.V.), sCR1/TP10 (Avant Immunotherapeutics), CAB-2/MLN-2222 (Millenium Pharmaceuticals), ofatumumab, a human monoclonal antibody that specifically binds the CD20 protein (also known as HuMax-CD20; Genmab A/S), a C3 inhibitor peptide and its functional analogs (Compstatin/POT-4; Potentia Pharmaceuticals, Inc.), a C5a receptor antagonist (PMX-53; Peptech, Ltd.), rhMBL (Enzon Pharmaceuticals), Factor D inhibitor BCX1470, sCR1-sLeX (a soluble from of CR1 that has been modified by the addition of sialyl Lewis x (sLe.sup.x) carbohydrate side chains yielding sCR1sLe (TP-20; Avant Immunotherapeutics, Inc.), APT070, which consists of the first three short consensus domains of human complement receptor 1, manufactured in recombinant bacteria and modified with a membrane-targeting amphiphilic peptide based on the naturally occurring membrane-bound myristoyl-electrostatic switchpeptide (Mirococept (Inflazyme Pharmaceuticals), TNX-234 (Tanox), TNX-558 (Tanox), an antibody or functional fragment thereof that binds factor B (TA106; Taligen Therapeutics, Inc.), an antibody that specifically binds the C5 receptor (e.g., neutrazumab; G2 Therapies, Inc.), Anti-properdin (Novelmed Therapeutics), HuMax-CD38 (Genmab A/S), a pegylated aptamer-based C5 inhibitor (ARC1905; Archemix, Inc.), and a small molecule/peptidomimetic antagonist for the C5a receptor protein (e.g., JPE-1375, JSM-7717; Jerini, Inc.), OmC1 protein, compstatin and its functional analogs, C1q inhibitors, C1 Inhibitor, C1r inhibitors, C1s inhibitors, analogues of sCR1, anti-C5a receptor antibodies and small-molecule drugs, anti-C3a receptor antibodies and small-molecule drugs, anti-C4a antibodies and their functionally equivalent fragments, anti-C6, C7, C8, or C9 antibodies, anti-Factor D antibodies, anti-properdin antibodies, Membrane Cofactor Protein (MCP), Decay Accelerating Factor (DAF), and MCP-DAF fusion protein (CAB-2), C4bp, Factor H, Factor I, Carboxypeptidase N, vitronectin and clusterin, CD59, c5a receptor antagonists, F-[oPdChaWR], and inhibitors of CD21, including but not limited to, antibodies, small molecule inhibitors, aptamers, nanobodies. In some embodiments, the complement inhibitor compound comprises eculizumab.

In some embodiments, the complement inhibitor is an antibody, antisense RNA, cDNA, small molecule, fusion protein, peptide, oligonucleotide.

A “small molecule” herein is defined as having a molecular weight below about 500 Daltons.

The term “cDNA” refers to complementary DNA. cDNA is a non-naturally occurring DNA that can be synthesized or manufactured from an messenger RNA (mRNA) template.

The term “fusion protein” (or chimeric protein) refers to a protein that can be created through the joining of two or more genes that originally encoded for separate proteins.

The term “antisense RNA” as used herein refers to the use of an RNA nucleotide sequence, complementary by virtue of Watson-Crick base pair hybridization, to a specific mRNA to inhibit its expression and then induce a blockade in the transfer of genetic information from DNA to protein. The antisense RNA molecule can be complementary to a portion of the coding or noncoding region of an RNA molecule, e.g., a pre-mRNA or mRNA. An antisense RNA can be, for example, about 10 to 25 nucleotides in length. An antisense RNA molecule can be constructed using chemical synthesis and/or enzymatic ligation reactions using procedures known in the art. Alternatively, the antisense RNA molecule can be transcribed biologically using an expression vector into which a nucleic acid has been subcloned in an antisense orientation (i.e., RNA transcribed from the inserted nucleic acid will be of an antisense orientation to a target nucleic acid of interest).

The term “peptide” refers to chains of amino acids linked by peptide bonds. Peptides can be distinguished from proteins on the basis of size and can contain, for example, approximately 50 or fewer amino acids. However, it will be understood that peptides can be greater than 50 amino acids as well. A multitude of peptides are known. Peptides can be classified or categorized according to their sources and function. Peptides can include plant peptides, bacterial/antibiotic peptides, fungal peptides, invertebrate peptides, amphibian/skin peptides, venom peptides, cancer/anticancer peptides, vaccine peptides, immune/inflammatory peptides, brain peptides, endocrine peptides, ingestive peptides, gastrointestinal peptides, cardiovascular peptides, renal peptides, respiratory peptides, opiate peptides, neurotrophic peptides, and blood-brain peptides.

The term “inhibition” or “inhibit” refers to a reduction of activity. By “inhibit” it is meant that the effect of the classical pathway of complement activation, specifically the formation of protein/heparin complexes, is reduced. The ability of a molecule to reduce the effect of the classical complement pathway can be determined by standard assays known in the art. The presence of a complement inhibitor molecule of the present disclosure can reduce complement activation by protein/heparin complexes by at least 20% (e.g., by at least 25%, 30%, 40%, 50%, 60%, 70% or 80% or more) compared to a control in the absence of a complement inhibitor molecule.

According to the methods of the present disclosure, the complement inhibitor(s) can be administered in a pharmaceutically acceptable composition. The term “pharmaceutically acceptable carrier” as used herein, includes genes, polypeptides, antibodies, liposomes, polysaccharides, polylactic acids, polyglycolic acids and inactive virus particles or indeed any other agent provided that the excipient does not itself induce toxicity effects or cause the production of antibodies that are harmful to the individual receiving the pharmaceutical composition. Pharmaceutically acceptable carriers may additionally contain liquids such as water, saline, glycerol, ethanol or auxiliary substances such as wetting or emulsifying agents, pH buffering substances and the like. Excipients may enable the pharmaceutical compositions to be formulated into tablets, pills, dragees, capsules, liquids, gels, syrups, slurries, suspensions to aid intake by the patient. A thorough discussion of pharmaceutically acceptable carriers is available in Remington's Pharmaceutical Sciences (Mack Pub. Co., N.J. 1991).

According to the methods of the present disclosure, the compositions can be administered by injection by gradual infusion over time or by any other medically acceptable mode. Administration can be, for example, intravenous, intraperitoneal, intramuscular, intracavity, subcutaneous, transdermal, or oral administration. Preparations for parenteral administration includes sterile aqueous or nonaqueous solutions, suspensions and emulsions. Examples of nonaqueous solvents are propylene glycol, polyethylene glycol, vegetable oil such as olive oil, an injectable organic esters such as ethyloliate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers, (such as those based on Ringer's dextrose), and the like. Preservatives and other additives can also be present such as, for example, antimicrobials, antioxidants, chelating agents, and inert gases and the like. Those of skill in the art can readily determine the various parameters for preparing these alternative pharmaceutical compositions without resorting to undue experimentation. When the compositions of the invention are administered for the treatment of pulmonary disorders the compositions can be delivered for example by aerosol.

Another aspect of the present disclosure provides a method of determining the presence of complement activation by protein/heparin binding complexes in a subject, the method comprising: (a) obtaining a biological sample from the subject; (b) determining the presence of plasma IgM in the biological sample; (c) if the plasma IgM is determined in an amount greater than the control, administering to the subject a therapeutically effective amount of a compound capable of blocking the classical pathway of complement activation such that complement activation by protein/heparin complexes is blocked in the subject. In some embodiments, the protein of the protein/heparin binding complex comprises platelet factor 4 (PF4).

Determining the presence of plasma immunoglobulins (e.g., IgM, IgG, IgA, IgE, IgD) in a biological sample can be achieved by methods described herein and known in the art that include, but are not limited to, rate nephelometry, mass spectroscopy, ELISA assay, or gel electrophoresis.

In some embodiments, plasma immunoglobulin (e.g., IgM) can be present in the biological sample in an amount that is greater than about 2%, 5%, 10%, 15%, 20%, 25%, 30%, 35%, 40%, 45%, 50%, 55%, 60%, 65%, 70%, 75%, 80%, or more than the amount of plasma immunoglobulin (e.g., IgM) in a control sample. In other embodiments, plasma immunoglobulin (e.g., IgM) can be present in the biological sample in an amount that is greater than about 2 fold, 5 fold, 10 fold, 15 fold, 20 fold, or more than the amount of plasma immunoglobulin (e.g., IgM) in a control sample.

In some embodiments, IgM can be present in the biological sample in an amount that is at or above about 200 μg/mL (e.g., about 250 μg/mL, about 300 μg/mL, about 350 μg/mL, about 400 μg/mL, about 450 μg/mL, or about 500 μg/mL, or about 600 μg/mL, or about 700 μg/mL, or about 800 μg/mL, or about 900 μg/mL, or about 1000 μg/mL, or about 1100 μg/mL, or about 1200 μg/mL, or about 1300 μg/mL, or about 1400 μg/mL, or about 1500 μg/mL, or about 1600 μg/mL, or about 1700 μg/mL, or about 1800 μg/mL, or about 1900 μg/mL, or about 2000 μg/mL, or about 2100 μg/mL, or about 2200 μg/mL, or about 2300 μg/mL, or about 2400 μg/mL, or about 2500 μg/mL, or about 2600 μg/mL, or about 2700 μg/mL, or about 2800 μg/mL, or about 2900 μg/mL, or about 3000 μg/mL, or greater). In some embodiments, IgM can be present in the biological sample in an amount between 200 μg/mL to about 3000 μg/mL.

As used herein, the term “biomarker” refers to naturally occurring biological molecule present in a subject at varying concentrations that is useful in predicting the risk or incidence of a disease or a condition, such as HIT. The biomarker can include nucleic acids, ribonucleic acids, or a polypeptide used as an indicator or marker for protein/heparin complexes in a subject. In some embodiments, the biomarker is a protein or an immunoglobulin (e.g., IgM). A biomarker can also comprise any naturally or non-naturally occurring polymorphism (e.g., single-nucleotide polymorphism [SNP]) present in a subject that is useful in predicting the risk or incidence of a disease, disorder, condition. For example, heparin pre-exposure levels of plasma IgM can constitute a stable biomarker for the risk of sensitization and possible development of HIT

Compounds useful in the methods are described herein and include variations of their pharmaceutically acceptable forms, including isomers such as diastereomers and enantiomers, salts, solvates, and polymorphs, as well as racemic mixtures and pure isomers of the compounds described herein, where applicable.

Since binding of complement coated antigen to B cell CD21 is highly relevant for subsequent immune activation, the findings described here are relevant to patients exposed to protein/heparin complexes. Blocking of classical pathway of complement activation can prevent antibody generation in diseases such as heparin induced thrombocytopenia (HIT).

Hence, another aspect of the present disclosure provides a method of treating a disease characterized by the onset of antibody generation or preventing the onset of a disease characterized by the onset of antibody generation in a subject, the method comprising, consisting of, or consisting essentially of administering to the subject a therapeutically effective amount of a compound capable of blocking the classical pathway of complement activation such that antibody generation is blocked in the subject and the onset of disease is prevented or the disease is treated.

As used herein, “treatment,” “therapy” and/or “therapy regimen” refer to the clinical intervention made in response to a disease, disorder or physiological condition manifested by a patient or to which a patient can be susceptible. The aim of treatment can include the alleviation or prevention of symptoms, slowing or stopping the progression or worsening of a disease, disorder, or condition and/or the remission of the disease, disorder or condition.

The term “disease” as used herein includes, but is not limited to, any abnormal condition and/or disorder of a structure or a function that affects a part of an organism. It can be caused by an external factor, such as an infectious disease or an antigen, or by internal dysfunctions, such as cancer, cancer metastasis, and the like. In some embodiments, the disease involves the classical complement pathway whereby inflammation, formation of protein/antibody complexes, and/or cellular injury results from the activation of the classical complement pathway. In some embodiments, the disease can be heparin-induced thrombocytopenia (HIT), protamine/heparin induced thrombocytopenia, or exogenously administered charged-particulate antigens.

In some embodiments, the disease can be heparin-induced thrombocytopenia (HIT). Heparin-induced thrombocytopenia (HIT) is an immune mediated pro-thrombotic disorder caused by antibodies to ultra-large complexes (ULCs) of platelet factor 4 (PF4) and heparin (H).

The term “onset of disease” as used herein refers to the first appearance of the signs or symptoms of an illness such as, for example, the onset of HIT. The onset of HIT can be characterized by pain, redness, and swelling in an arm or leg, bruises on the skin, a rash or sore at the site that a heparin shot was administered to a subject, and weakness, numbness, or problems moving extremities (e.g., arms or legs).

Yet another aspect of the present disclosure provides a method of treating heparin-induced thrombocytopenia (HIT) in a subject, the method comprising, consisting of, or consisting essentially of administering to the subject a therapeutically effective amount of a compound capable of blocking the classical pathway of complement activation such that the HIT is treated in the subject.

In another embodiment, the treatment methods described herein further comprise administering to the subject a second or further treatment regimen and/or administration of additional therapeutic agents, in combination with the complement inhibitor compound.

Yet another aspect of the present disclosure provides all that is disclosed and illustrated herein.

The following examples are offered by way of illustration and not by way of limitation.

### EXAMPLES

Materials and Methods

Materials: Recombinant human platelet factor 4 (PF4) was purified as described (Rauova L, et al. (2010) Blood, 116(23):5021-5031). UFH was from Elkins-Sinn Inc., Cherry Hill, N.J. Unless specified, other chemicals, buffers and tissue culture reagents were purchased from Millipore Sigma (St. Louis, Mo.). IgM and IgG from healthy donor plasma and IgM from myeloma patient plasma was purchased from Athens Research and Technology (Athens, Ga.). Intravenous immunoglobulin (IVIG) was purchased from Grifols (Los Angeles, Calif.). The following antibodies were used: anti-C1q (Cell Sciences, Inc., Newburyport, Mass.), anti-C3c (Quidel, San Diego, Calif.), anti-MBL (R&D Systems, Minneapolis, Minn.) and murine IgG1 isotype control (Invitrogen, Carlsbad, Calif.), fluorescently conjugated anti-human CD19 and conjugated streptavidin (eBioscience, San Diego, Calif.), fluorescently conjugated goat anti-human IgM (Jackson Labs, Westgrove, Pa.). Monoclonal antibody KKO (IgG2_{b}κ recognizing PF4/heparin), ADA (IgG3 recognizing protamine sulfate (PRT)/heparin) and 2E4 (monoclonal IgM with polyreactive specificities to single-stranded DNA, β-galactosidase and other antigens) were developed, purified and isolated in the laboratory according to published methods. PF4/heparin-specific IgM was isolated using beads coated with PF4 bound to heparin immobilized on diamino-dipropylamine agarose (ThermoFisher Scientific, Waltham, Mass.), as previously described.

Blood Samples: Blood from healthy donors or patients receiving heparin therapy was collected into citrate with written consent using an IRB approved protocol (Duke IRB#: Pro00010740). Human subjects were enrolled in accordance with the Declaration of Helsinki. Human umbilical cord blood was obtained as discarded clinical samples under an IRB exempt provision (Duke IRB#: Pro00047355). Where indicated, studies were performed in whole blood or 100% plasma from healthy donors.

Immunoglobulin Levels: Total immunoglobulins (IgG, IgA and IgM) in serum were quantified by the Duke University Hospital Clinical Immunology Lab by rate nephelometry.

Antigen-C3c capture ELISA assay and Specificity ELISA: Antigen-specific monoclonal antibodies (mouse anti human PF4/heparin; KKO or mouse anti-protamine/heparin; ADA at 2 μg/mL) were incubated overnight on a microtiter plate (in phosphate buffered saline, PBS) followed by washing and blocking with 1% bovine serum albumin (BSA) in PBS for 2 hours. To activate complement, plasma was incubated with buffer or heparin alone or PF4±heparin or PRT±heparin at 37° C. for 1 hour (hr) followed by addition of 10 mM ethylenediaminetetraacetic acid (EDTA) to inhibit further complement generation. Unless specified antigen concentrations were 25 μg/mL (PF4) and 0.25 U/mL (heparin) for KKO coated plates and 125 μg/mL (PRT) and 6 U/mL (heparin) for ADA coated plates.

Next, plasma containing antigen fixed by complement activation fragments was added to the capture plate for 1 hr followed by serial washes. Complement-coated antigen was detected using a biotinylated anti-C3c antibody (recognizes C3 and all C3c-containing fragments of C3, including iC3b; Quidel Corporation, San Diego, Calif.) followed by colorimetric detection as previously described.

For studies involving immunoglobulins (IgM, IgG, myeloma IgM, monoclonal polyreactive IgM), ethylenediaminetetraacetic acid (EDTA), ethylene glycol-bis (β-aminoethyl ether)-N,N,N′,N′-tetraacetic acid (EGTA), Magnesium chloride (MgCl₂) or classical/lectin pathway, reagents were added to plasma before incubating with antigen.

Antigen specificity was determined against various antigens (bovine serum albumin (BSA), PF4, PF4/heparin, PRT, PRT/heparin, lysozyme (Lys), Lys/heparin or heparin alone) using protein concentrations of 10 μg/mL and heparin (0.2-1.3 U/mL, based on optimal antigenic protein:heparin ratios, PHR) by ELISA. Bound IgM was detected with goat anti-human IgM (μ-chain specific) peroxidase conjugated antibody by colorimetric detection.

Flow cytometry studies: Flow-based studies of antigen, complement, or IgM binding to B cells were performed as described previously (Khandelwal S, et al. (2016) Blood, 128(14):1789-1799). Patient samples were processed without addition of exogenous antigen. Cells were analyzed using a BD FACS Canto Flow cytometer (BD Biosciences, Franklin Lakes, N.J.). Signals from a minimum of 10,000 cells were acquired from each sample. Analyses were performed using FCS express software (Version 5 Flow Research Edition; De Novo Software).

IgM studies: Goat anti-human IgM (μ-chain specific) agarose beads were used to deplete IgM from plasma. Anti-IgM or control agarose beads were incubated with plasma and depleted plasma supernatant was analyzed in the antigen-C3c capture assay described above.

Proteomic analysis: Plasma from donors identified as having a “high”, “intermediate” or “low” complement activity in the antigen-C3c capture ELISA assay were submitted to the Duke Proteomics and Metabolomics Shared Resource (https://genome.duke.edu/cores-and-services/proteomics-and-metabolomics). Proteomic data was analyzed using published protocols. (Reidel B, et al. (2011) Mol. Cell. Proteomics., 10(3):M110.002469) Principal component analysis, statistical tests and agglomerative clustering was performed using the Rosetta Elucidator.

Statistics: Data are expressed as mean±standard deviation (SD). Significance was calculated using Student's t-test or one-way ANOVA. Descriptive statistics (means/standard deviations/range are used to describe continuous variables. Normality was assessed using the Shapiro-Wilk/Anderson-Darling tests. Correlations between normally distributed continuous variables are conducted using Pearson correlations, and correlations between non-normally distributed variables are conducted using Spearman correlations. Statistical significance is assessed at alpha=0.05. No adjustment is made for multiple testing. Statistical analyses were performed using the SAS 9.4 statistical software (SAS Institute Inc., Cary, N.C.) or GraphPad Prism (Graph Pad Software Version 7.0).

### Example 1

### Complement Responses to PF4/Heparin Complexes Among Healthy Donors Defines a Donor “Phenotype”

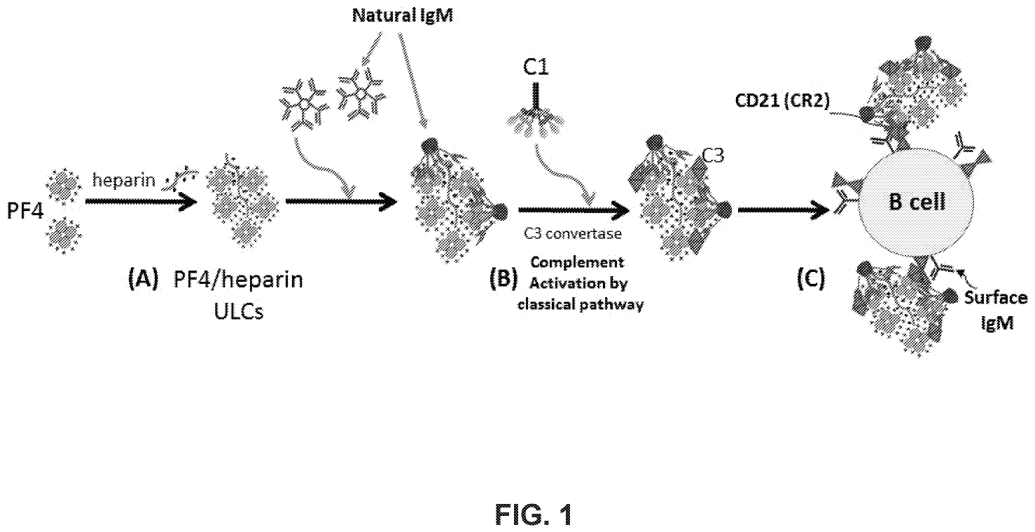

To examine the mechanism underlying C′ activation by PF4/heparin complexes (see FIG. 1), a plasma-based capture immunoassay to detect complement activation fragment C3c bound to PF4/heparin complexes was developed. In this assay, when plasma from healthy donors is incubated under identical conditions with buffer, PF4 alone or PF4/heparin, the degree of C′ activation, as determined by antigen-bound C3c, varied significantly among healthy donors.

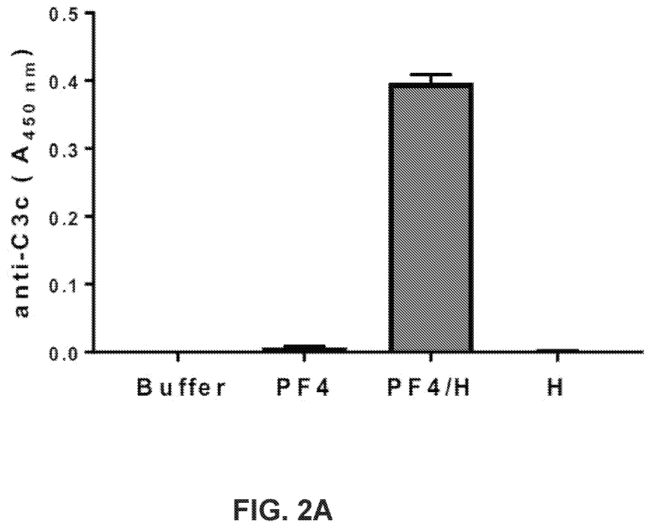

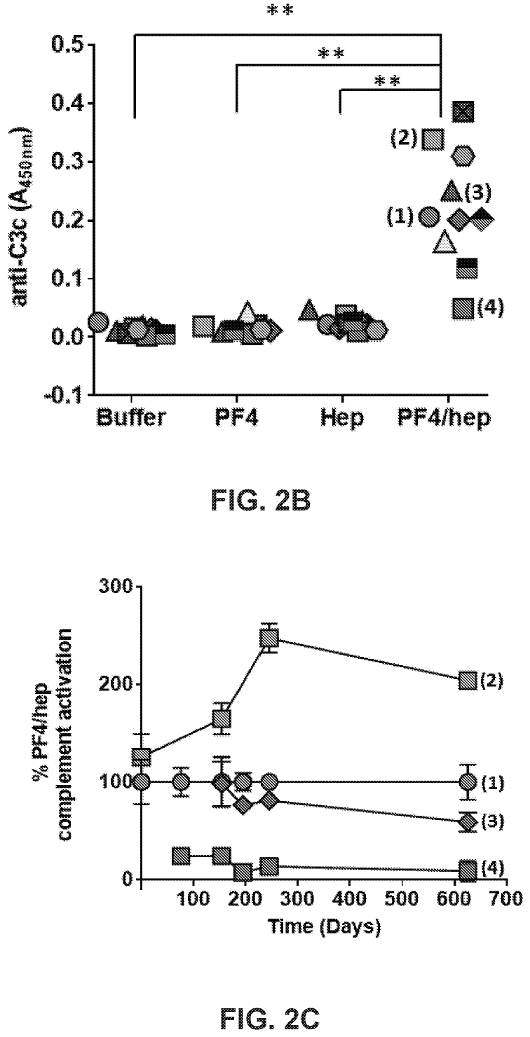

Plasma from a healthy donor was incubated with buffer or antigen (PF4, 25 μg/mL±heparin) or heparin alone for 60 minutes at 37° C. followed by addition of 10 mM EDTA to inhibit further C activation. After C′ activation, C′ activation was detected by a capture immunoassay using KKO and an anti-C3c antibody. The binding of C3c to the PF4/heparin complexes was determined by ELISA based antigen capture assay. As shown in FIG. 2A, ELISA based antigen capture assay detects C′ activation by PF4 heparin.

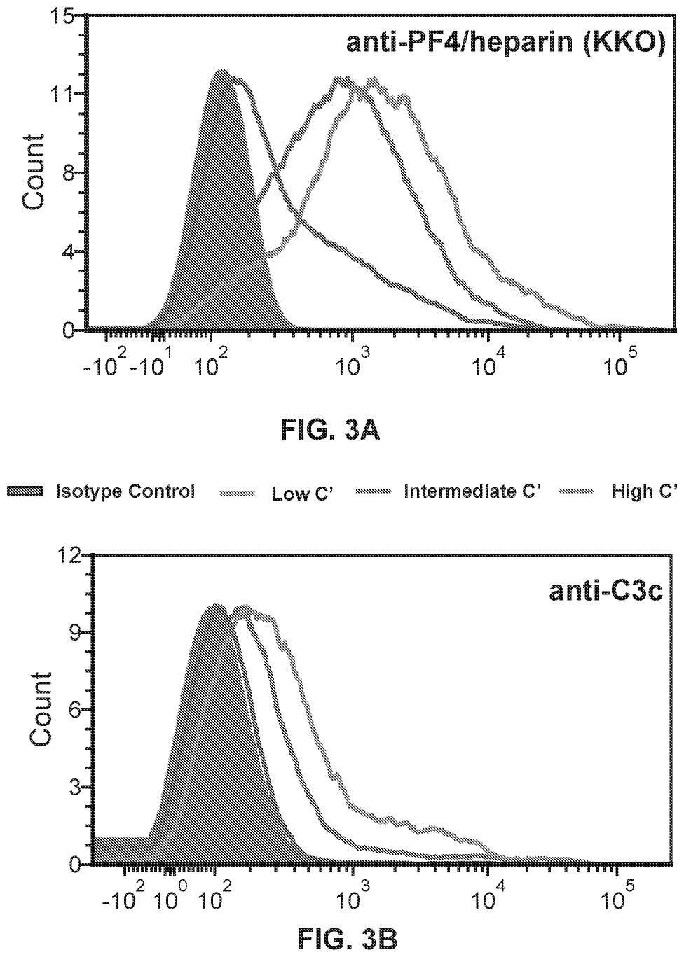

As shown in FIG. 2B, complement activation in this assay occurs when plasma from healthy donors with no prior history of heparin exposure (n=10) was incubated with PF4/heparin but not with buffer or PF4 alone or heparin alone and binding of C3c to PF4/heparin complexes was measured by antigen-C3c capture ELISA assay. Complement activation in response to PF4/heparin was highly variable, with some donors expressing high reactivity (e.g. donor “2”), while others expressed intermediate (e.g. donors “1” and “3”) and/or low (e.g. donor “4”) levels of complement activation. Intriguingly, for a given donor, responses to PF4/heparin constituted a “stable” phenotype that remained high, intermediate or low over time (up to ˜1.7 years; FIG. 2C). Differences in complement activation among “high”, “intermediate” or “low” responders correlated with the amount of PF4/heparin antigen and C3c deposited on B cells. Whole blood from “high”, “intermediate” and “low” complement (C′) response type healthy donors from FIG. 2B and FIG. 2C was incubated with PF4 and heparin and binding of PF4/heparin and C3c to B cells was determined by flow cytometry as described in the materials and methods section. (FIGS. 3A and 3B). It was next determined whether these differences in donor phenotype are governed by circulating plasma components.

### Example 2

### Donor Phenotype Correlates with Plasma IgM Levels

To determine if phenotypic differences among donors were due to variability in levels of complement or complement regulatory proteins, the plasma proteome of healthy donors with high (n=3), intermediate (n=2) or low (n=3) C′ activation were examined. To determine the serologic basis for the donor “phenotype”, the plasma proteome of 8 donors displaying “high” (n=3), “intermediate” (n=2) or “low” (n=3) reactivity was examined by mass spectrometric analysis. As shown in Table 1, mass spectrometry identified five proteins that showed significant correlation with “high”/“intermediate” vs. “low” donors. (Table 1 showing highest to lowest significance in p-value for high/intermediate vs low): IgM μ chain C region (40 peptides quantified; 4-fold increase; p=0.001), complement C1q subcomponent subunit B (10 peptides quantified; 1-fold increase; p=0.01), complement factor H-related protein 4 (1 peptide quantified; 2-fold increase; p=0.031), complement C1q subcomponent subunit A (4 peptides quantified; 1-fold increase; p=0.033) and complement factor D (4 peptides quantified; 1-fold decrease; p=0.034). Based on data showing the greatest peptide coverage for IgM (40 peptides) and associated significant p-values, the correlation between complement activation with donor IgM was examined in more detail. A wide stable variation in complement activation was observed when PF4/heparin was added to plasma of healthy donors, indicating a responder “phenotype” (high, intermediate or low).

TABLE 1
The fold changes in the levels of various complement and
complement regulatory proteins in the high (n = 3)/intermediate
(n = 2) vs low (n = 3) complement responders.

| | | | Fold change | p-value (t-test) |
|---|---|---|---|---|
| | | # | High/ | High/ |
| Primary | | quantified | Intermediate | Intermediate |
| Protein Name | Protein Description | peptides | vs. Low | vs. Low |
| IGHM_HUMAN | Ig MU chain C region | 40 | 4.08 | 0.001 |
| C1QB_HUMAN | Complement C1q | 10 | 1.13 | 0.01 |
| | subcomponent subunit | | | |
| | B | | | |
| FHR4_HUMAN | Complement factor H- | 1 | 2.07 | 0.031 |
| | related protein 4 | | | |
| C1QA_HUMAN | Complement C1q | 4 | 1.12 | 0.033 |
| | subcomponent subunit A | | | |
| CFAD_HUMAN | Complement factor D | 4 | −1.19 | 0.034 |
| MBL2_HUMAN | Mannose-binding | 4 | −2.13 | 0.054 |
| | protein C | | | |
| CO9_HUMAN | Complement component | 22 | 1.49 | 0.131 |
| | C9 | | | |
| C1RL_HUMAN | Complement C1r | 6 | 1.53 | 0.133 |
| | subcomponent-like | | | |
| | protein | | | |
| FHR1_HUMAN | Complement factor H- | 7 | 2.69 | 0.134 |
| | related protein 1 | | | |
| FCN3_HUMAN | Ficolin-3 | 8 | −1.29 | 0.139 |
| IC1_HUMAN | Plasma protease C1 | 29 | 1.16 | 0.153 |
| | inhibitor | | | |
| C1QC_HUMAN | Complement C1q | 12 | 1.08 | 0.165 |
| | subcomponent subunit C | | | |
| CO4A_HUMAN | Complement C4-A | 4 | 2.25 | 0.208 |
| CO7_HUMAN | Complement component | 29 | −1.13 | 0.21 |
| | C7 | | | |
| CO2_HUMAN | Complement C2 | 23 | −1.13 | 0.229 |
| CFAI_HUMAN | Complement factor I | 25 | −1.15 | 0.24 |
| CO4B_HUMAN | Complement C4-B | 138 | 1.18 | 0.269 |
| C1S_HUMAN | Complement C1s | 24 | 1.05 | 0.36 |
| | subcomponent | | | |
| CFAH_HUMAN | Complement factor H | 108 | −1.07 | 0.392 |
| C1R_HUMAN | Complement C1r | 33 | 1.06 | 0.438 |
| | subcomponent | | | |
| CO8G_HUMAN | Complement component | 12 | −1.11 | 0.438 |
| | C8 gamma chain | | | |
| CO8B_HUMAN | Complement component | 25 | −1.06 | 0.442 |
| | C8 beta chain | | | |
| MASP1_HUMA | Mannan-binding lectin | 7 | −1.10 | 0.542 |
| | serine protease 1 | | | |
| CO3_HUMAN | Complement C3 | 235 | −1.07 | 0.603 |
| FHR5_HUMAN | Complement factor H- | 3 | −1.11 | 0.603 |
| | related protein 5 | | | |
| FHR2_HUMAN | Complement factor H- | 3 | 1.14 | 0.606 |
| | related protein 2 | | | |
| MASP2_HUMAN | Mannan-binding lectin | 1 | −1.17 | 0.647 |
| | serine protease 2 | | | |
| CLUS_HUMAN | Clusterin | 24 | 1.05 | 0.664 |
| VTNC_HUMAN | Vitronectin | 24 | −1.06 | 0.692 |
| FCN2_HUMAN | Ficolin-2 | 4 | −1.11 | 0.73 |
| CO8A_HUMAN | Complement component | 49 | −1.03 | 0.82 |
| | C8 alpha chain | 23 | | |
| CFAB_HUMAN | Complement factor B | 49 | 1.03 | 0.873 |
| CO5_HUMAN | Complement C5 | 88 | −1.00 | 1 |
| CO6_HUMAN | Complement component | 34 | 1.01 | 1 |
| | C6 | | | |

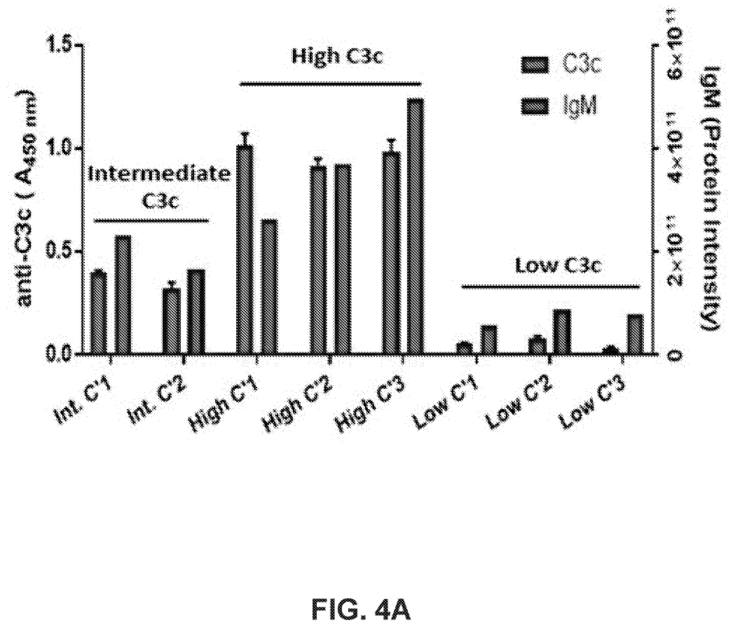

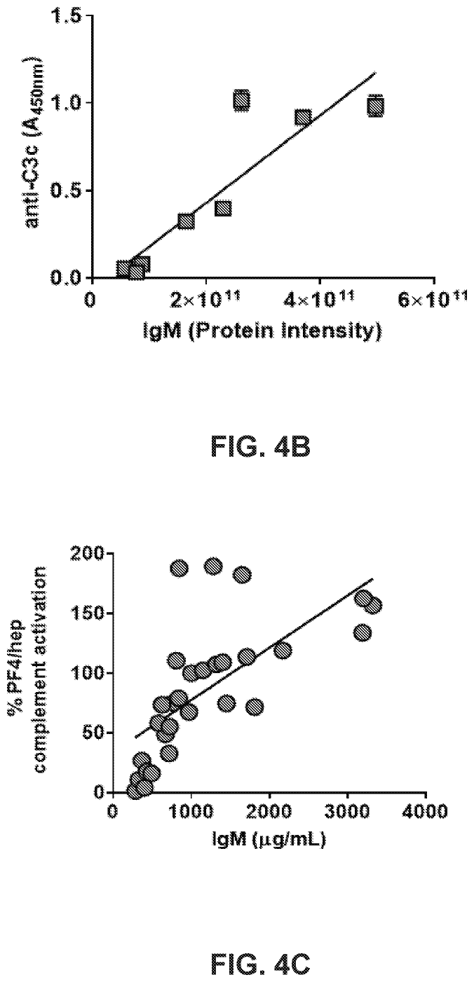

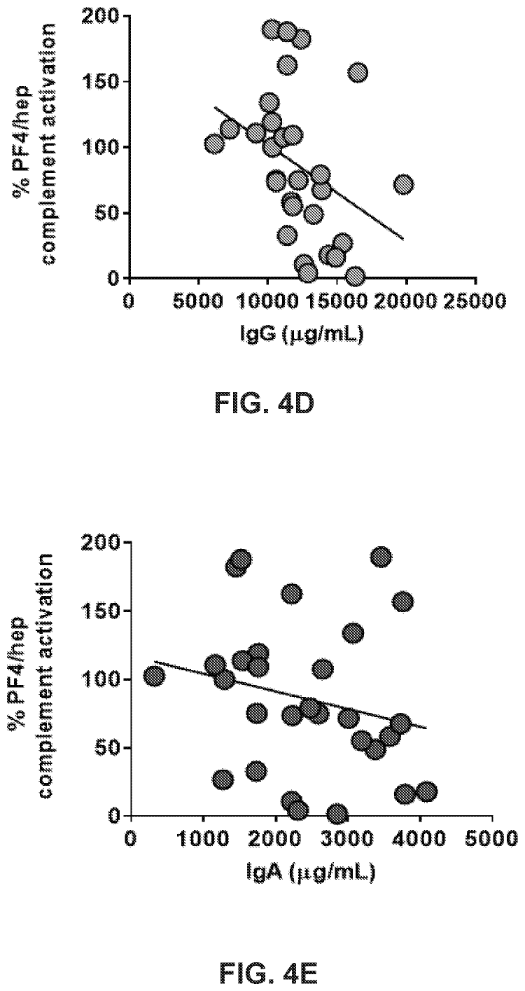

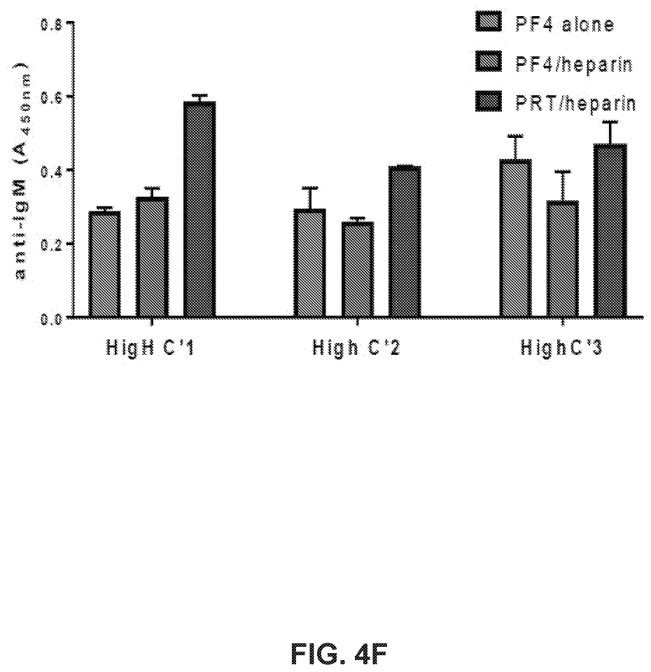

C′ activation phenotype donors was subjected to proteomic analysis. Proteomic analysis did not reveal differences in C′ or C′-regulatory proteins among donors tested. However, there was a marked correlation between donor phenotype and plasma IgM levels FIG. 4A shows the PF4/heparin induced C′ activation by different donors (determined by ELISA based antigen capture assay) and their plasma IgM levels (quantified by proteomic analysis).

As shown in FIG. 4B by mass spectrometry, an individual's IgM levels showed a strong correlation with complement activation (r=0.898, p<0.005; Pearson's correlation). FIG. 4A and FIG. 4B present the same data but represent the data differently.

To affirm these findings and examine the influence of other immunoglobulins, a larger cohort (n=29) of healthy individuals for complement activation response to PF4/heparin and measured corresponding IgM, IgG and IgA levels was tested in our clinical laboratory. As shown in FIGS. 4C-4E, there was again a strong correlation between complement activation by PF4/heparin and total IgM (0.82; p<0.0001; by Spearman correlation as IgM levels were not normally distributed) but not IgG or IgA levels in the same samples (IgG: −0.36; p=0.05; IgA: r=−0.22, p=ns; by Pearson's correlation for normally distributed data). Thus, there is a correlation between IgM and C′ activation with FIG. 4C that is not present with the data shown in FIG. 4D and FIG. 4E for IgG and IgA, respectively.

The antigen specificity of IgM from donors expressing high C′ activation phenotype was examined next. Binding of plasma IgM from high C′ phenotype donors (n=3) to different antigen was determined by ELISA on a microtiter plate coated with PF4 alone (10 μg/mL) or PF4 (10 μg/mL)+Heparin (0.4 U/mL) or Protamine sulfate (PRT; 31 μg/mL)+heparin (4 U/mL). As shown in FIG. 4F, IgM binding was not antigen-specific, as IgM bound to PF4/heparin as well as to PF4 alone or PRT/heparin complexes. Thus, IgM from high C′ phenotype donors shows binding to multiple antigens.

### Example 3

### Plasma IgM Mediates Complement Activation by PF4/Heparin Complexes

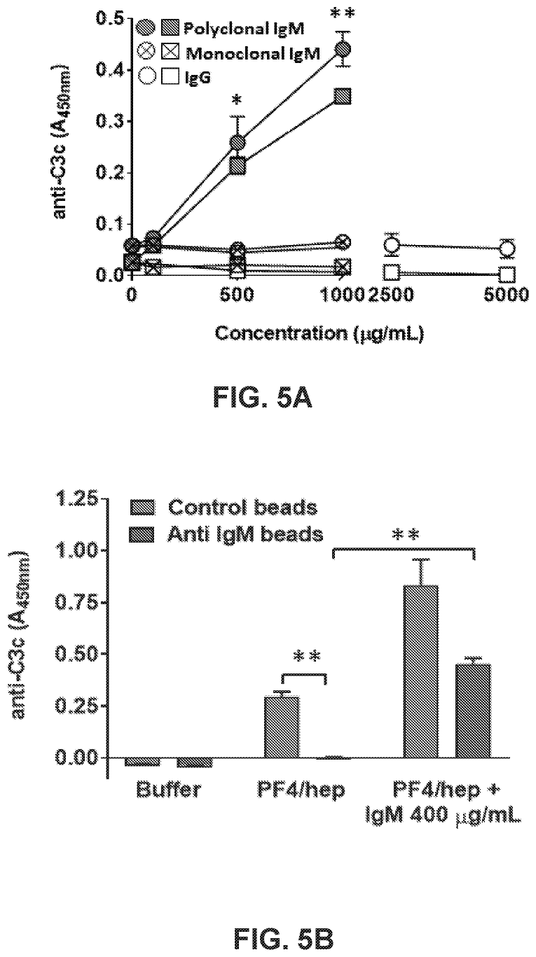

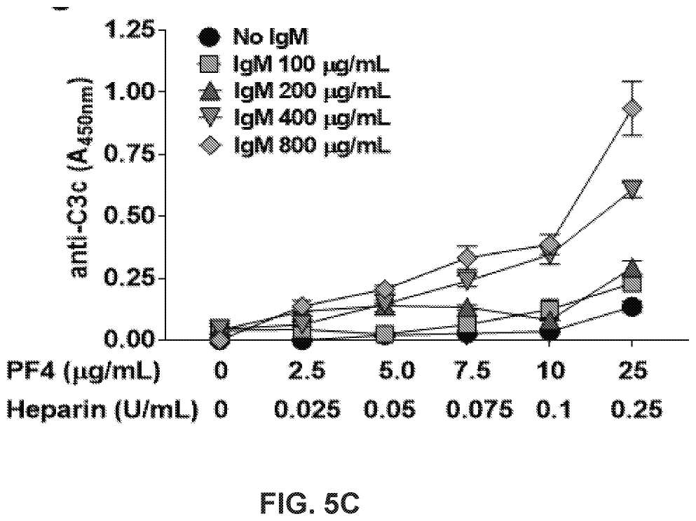

The studies shown in FIG. 4A-4F demonstrate a strong correlation between an individual's plasma IgM levels and the extent of complement activation response to PF4/heparin, but they do not show that IgM is required. To investigate the involvement of IgM, IgM was augmented or depleted from the plasma of donors with low or intermediate reactivity, respectively. C′ activation by IgM did not require antigen-specific IgM, as IgM from healthy donors reacted equally to microtiter plates coated with PF4 alone, protamine±H, Lysozyme+H and albumin. Addition of polyclonal commercial IgM (0-1000 μg/mL; isolated and pooled from ˜20 healthy donors) to the plasma of two “low phenotype” donors caused a dose-dependent increase in complement activation, but not monoclonal IgM (FIG. 5A, ˜10-fold increase in C3c generation seen with 1000 μg/mL IgM vs 0 IgM, p<0.0001). Neither polyclonal IgG (0-5000 μg/mL; FIG. 5A) nor monoclonal IgM (0-1000 μg/mL) restored complement activation at any concentration tested (FIG. 5A), even when higher IgG concentrations were tested to mimic the 5-10 fold higher levels of IgG in plasma.

Conversely, removal of IgM from plasma diminished C3c generation by PF4/heparin. Specifically, when plasma from an “intermediate phenotype” donor was depleted of IgM by using anti IgM beads and complement activation by PF4/heparin with or without adding commercial IgM (400 μg/mL), there was marked loss of complement activation in response to PF4/heparin (FIG. 5B, 4th column) as compared to same plasma treated with control beads (FIG. 5B p<0.0001, 3rd column). Furthermore, repleting IgM (400 μg/mL) in plasma devoid of IgM rescued complement activation by PF4/heparin (FIG. 5B p<0.0001 compared to no added IgM, 6th column). As expected, addition of similar amounts of IgM to plasma incubated with control beads enhanced complement activation (FIG. 5B).

Next, the concentrations of IgM and PF4/heparin were varied to mirror changes likely to occur in the clinical setting. To do so, plasma from a donor with low IgM levels was used and the effects of increasing IgM (0-800 μg/mL) and PF4/heparin concentrations (PF4 2.5-25 μg/mL and heparin 0.025-0.25 U/mL) at a fixed molar PHR of 6.6 were tested. As shown in FIG. 5C, complement activation was more dependent on changes in IgM levels than in PF4/heparin. At levels of IgM<200 μg/mL, complement activation was fairly insensitive to levels of PF4/heparin. In contrast, when the IgM was >400 μg/mL, lower PF4/heparin ratios (7.5:0.075 and 10:0.1, respectively) sufficed to activate complement. Together, these findings demonstrate that complement activation by PF4/heparin complexes is dependent on IgM.

Differences in circulating IgM levels can contribute to susceptibility towards C′ activation by PF4/heparin complexes and subsequent development of PF4/heparin antibodies in patients receiving heparin therapy. These findings indicate that targeting the classical pathway can be a strategy for preventing the development of HIT antibodies.

### Example 4

### Polyreactive, Naturally-Occurring IgM Mediates Complement Activation by PF4/Heparin

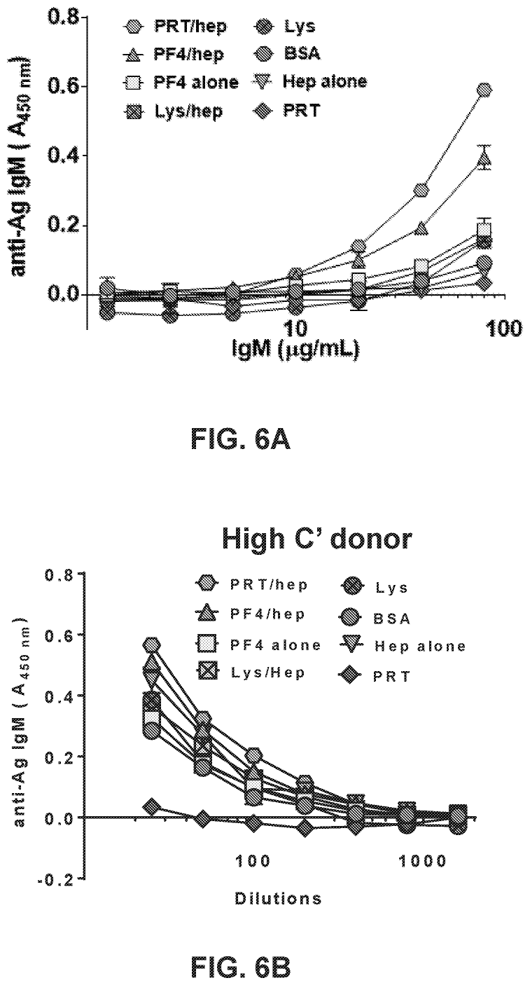

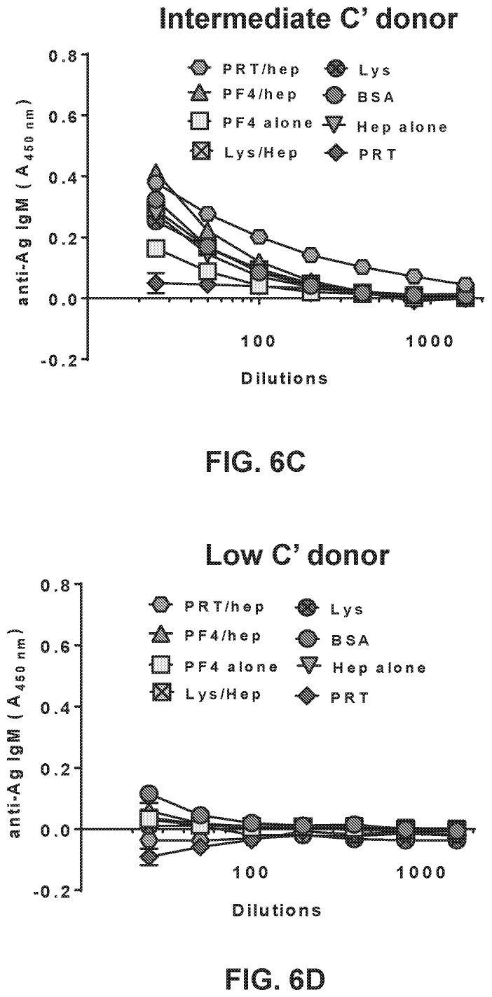

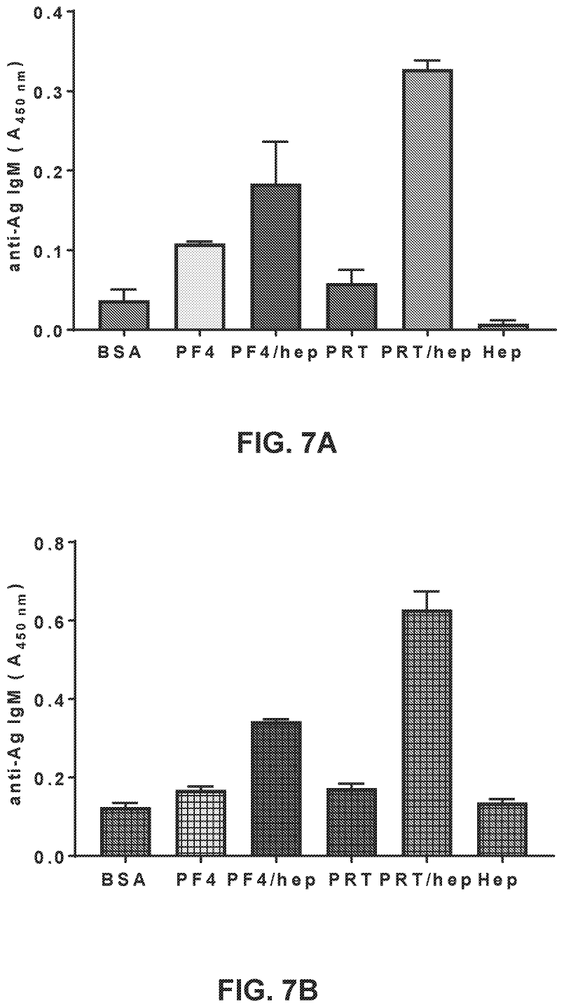

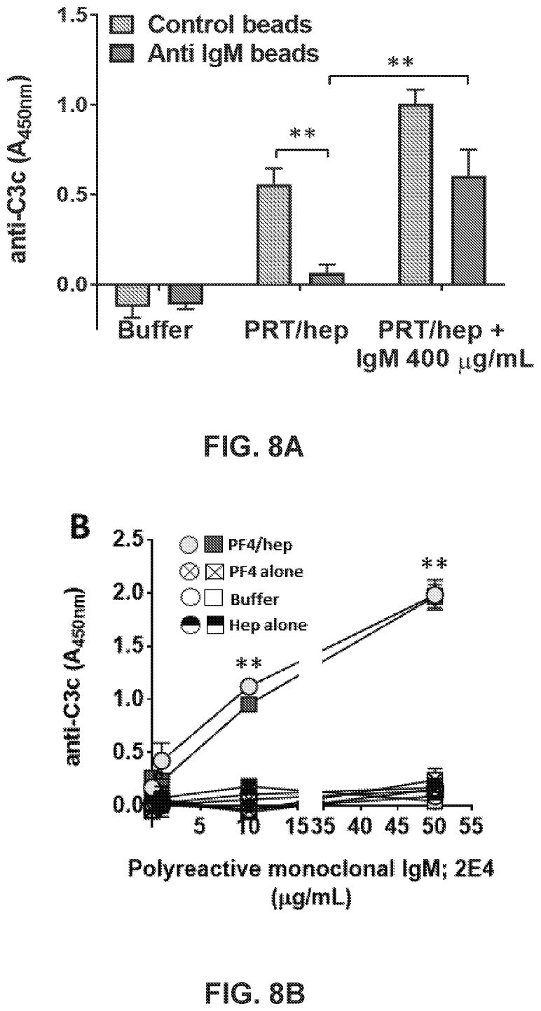

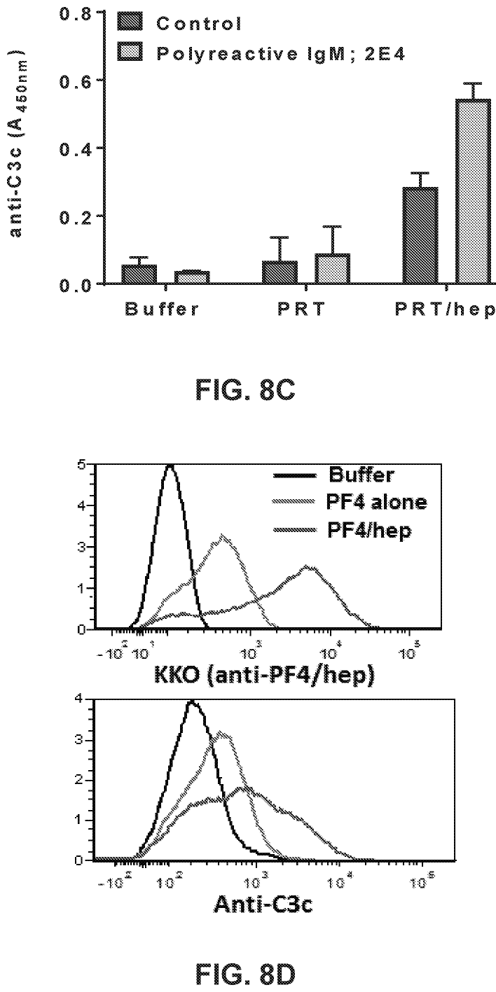

The findings that plasma containing IgM from individual healthy donors without prior heparin exposure (FIG. 2 and FIG. 4) and that pooled IgM from healthy donors (FIG. 5) activate complement in response to PF4/heparin suggests possible involvement of “natural” IgM. To investigate the role of natural IgM, the antigen-binding specificities of commercial donor IgM was examined, which can be assumed to reflect little to no contribution from individuals who have been exposed to heparin. Binding of pooled IgM (or plasma dilutions of individual donors) to a panel of heparin-binding proteins was measured in the presence or absence of added heparin. As seen in FIG. 6A, commercial IgM showed broad reactivity to a variety of antigens. In general, antigen reactivity was higher in the presence of heparin. Plasma from specific donors with high (FIG. 6B) and intermediate (FIG. 6C) IgM levels also showed broad reactivity with multiple antigens, but reactivity to individual antigens varied slightly from commercial IgM. The low IgM donor showed minimal reactivity to all antigens tested (FIG. 6D). To exclude a sub-population of PF4/heparin-specific IgM within a polyclonal IgM pool, commercial IgM was subjected to affinity purification using a PF4/heparin column. As shown in FIGS. 7A-7B, affinity purified IgM did not differ from unfractionated IgM with respect to binding various antigens, thus excluding the presence or role for antigen-specific IgM in complement activation. Moreover, polyreactive IgM also activated complement in the presence of PRT/heparin complexes. As shown in FIG. 8A, PRT/heparin complexes activated complement in plasma containing IgM, but not in plasma depleted of IgM. As with PF4/heparin, addition of polyclonal IgM to IgM depleted plasma restored complement activation (FIG. 8A).

Two independent approaches were used to confirm or exclude existence of natural IgM. First, the complement activating activity of a monoclonal IgM antibody, 2E4, with broad specificities analogous to natural IgM was examined. 2E4 recognizes diverse endogenous antigens (e.g., single stranded DNA β-galactosidase, insulin) and several strains of streptococci. Addition of 2E4 to the plasma of two donors with low complement reactivity initiated an antibody-dependent increase in complement activation in the presence of PF4/heparin (p<0.0001 at 10 and 50 μg/mL 2E4) but not following addition of PF4 alone, heparin alone or buffer (FIG. 8B). As with PF4/heparin, 2E4 activated complement in response to PRT/heparin complexes, but not when plasma was incubated with PRT alone (FIG. 8C). Next, complement activation in response to PF4/heparin in cord blood, which is enriched in natural IgM was examined. Because IgM levels in cord blood are <10% of adult levels, which is insufficient to activate complement in the plasma-based C3c immunoassay, we used the more sensitive flow-based assay. Cord blood incubated with PF4/heparin increased antigen (FIG. 8D, top panel) and C3c (FIG. 8C, bottom panel) on B cells (third line) compared to incubation with PF4 (second line) or buffer alone (first line). These data further support the concept that naturally occurring IgM mediates complement activation by PF4/heparin complexes.

A major finding from the studies described herein is that non-immune or “natural” IgM, rather than immune IgM, is likely responsible for PF4/heparin-mediated complement activation. First, plasmas from healthy donors with no prior heparin exposure activate complement in an IgM-dependent manner (FIGS. 2-3 and 5). In individuals with low IgM, polyclonal commercial IgM (derived from the plasma of ˜20 healthy donors), but not monoclonal IgM derived from a myeloma patient's plasma or IgG (FIG. 5A) enhanced the complement activation response to PF4/heparin. Commercial IgM and individual donor IgM display broad reactivity with multiple unrelated antigens (FIGS. 6A and 6B). Moreover, a monoclonal antibody with polyreactivity (pAb2E4) to bacterial antigens (FIG. 8B) as well as cord blood plasma (FIG. 8D), which contains mostly natural IgM, activates complement in response to PF4/heparin as well. Thus, these data indicate that polyreactive IgM that develops in response to early encounters with endogenous or exogenous antigens with features of PF4/heparin-like molecules likely contribute to host immunity allowing for rapid development of antigen-specific antibodies that mediate HIT.

The studies described herein can also help to reconcile seemingly disparate observations on the contributions of innate and adaptive immunity to the development of HIT antibodies. Whereas several studies, using athymic mice and mice depleted of CD4+ T cells indicate an absolute requirement for T cells, other studies show T cell independence. In these latter studies, mice lacking marginal zone (MZ) B cells, B lymphocytes belonging to the innate immune system (Cerutti A, et al. (2013) Nat Rev Immunol., 13(2):118-132), were unable to develop antibodies after PF4/heparin immunization. These findings led the authors to speculate that HIT was likely T cell independent. However, as MZ B cells are an important source of polyreactive IgM necessary for complement activation, antigen transport and subsequent adaptive immunity, the requirements for MZ B cells are in keeping with their role in bridging innate and adaptive immunity.

### Example 5

### Complement Activation by IgM is Mediated Through the Classical Pathway

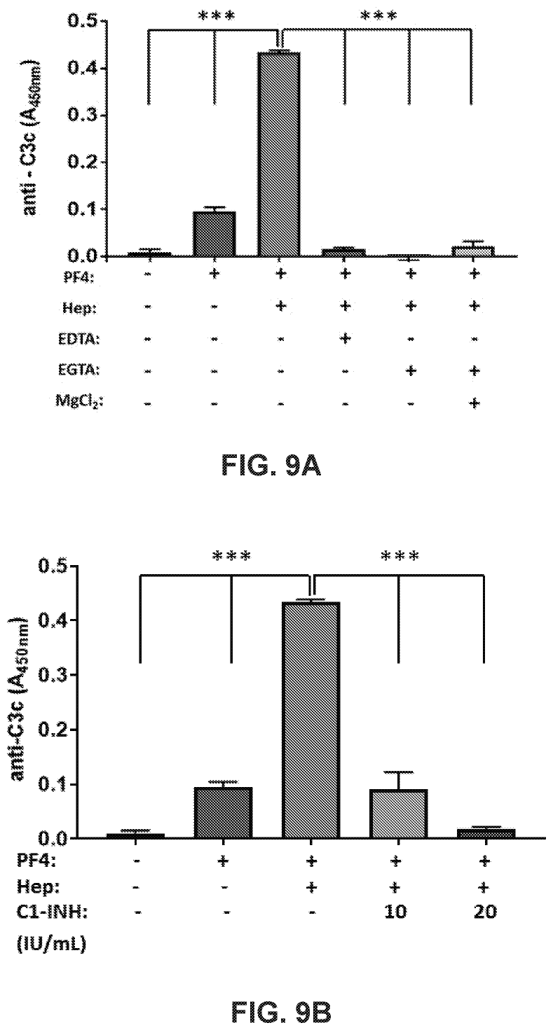

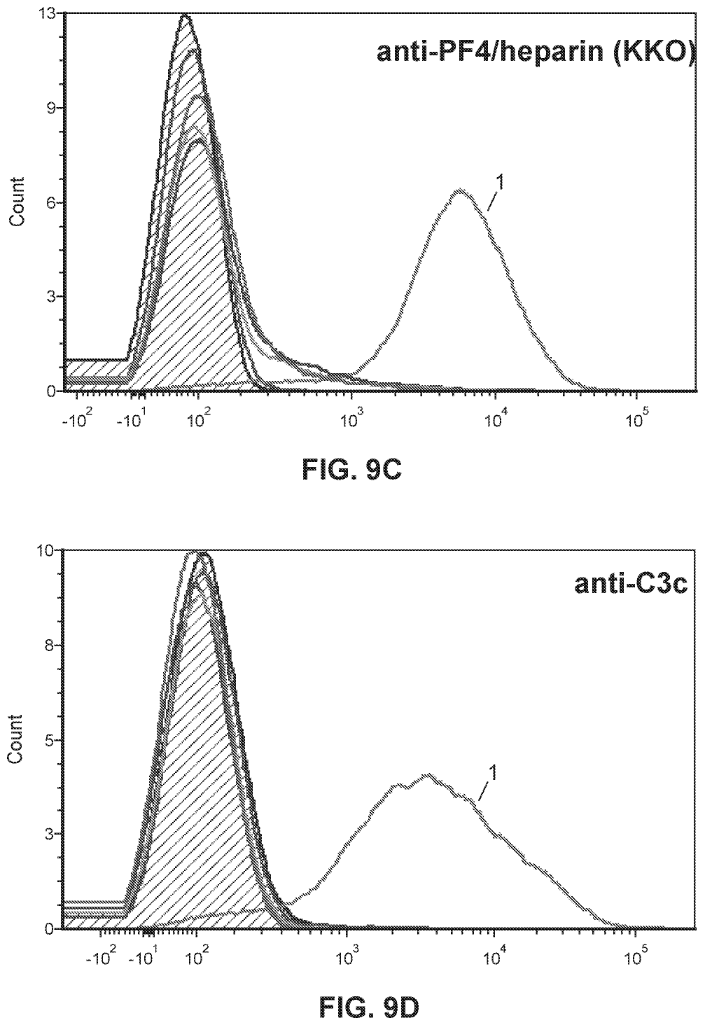

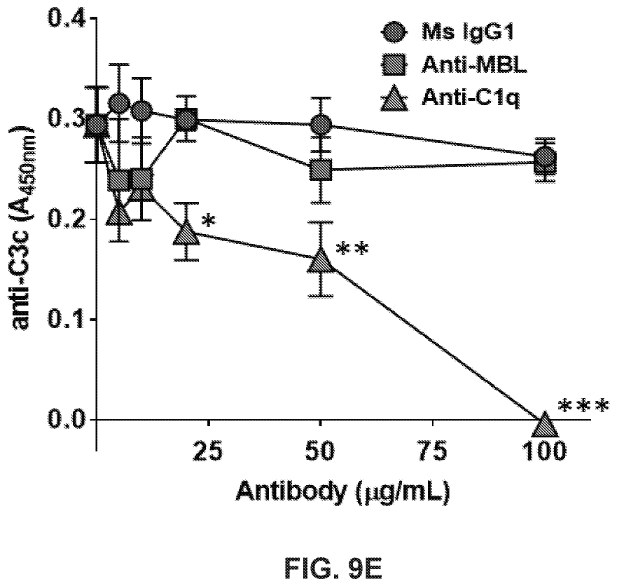

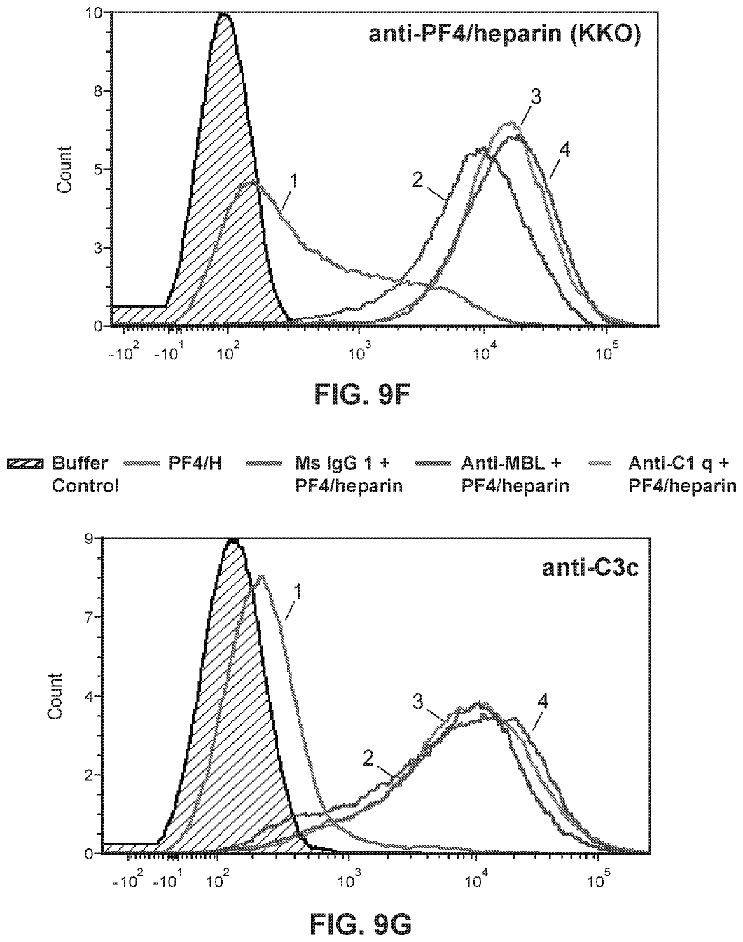

The complement system can be activated by the alternative, classical, and/or lectin pathways. To first investigate the alternative pathway in PF4/heparin-mediated complement activation, differential chelation studies using EDTA and EGTA was performed, wherein the alternative pathway, sensitive to Mg²⁺, is inhibited by EDTA, but not EGTA. As shown in FIG. 9A, addition of EDTA or EGTA to plasma prior to addition of PF4/heparin eliminated complement activation. Further, Mg²⁺ supplementation of EGTA-treated plasma did not rescue complement activation by PF4/heparin. Plasma from a healthy donor was incubated with or without C1-inhibitor (10 and 20 IU/mL) before incubating with PF4/heparin and complement activation by PF4/heparin was determined by antigen-C3c capture ELISA assay. As shown in FIG. 9B, complement activation was reduced using C1 esterase inhibitor. Similar results were obtained in whole blood assay using flow cytometry (FIG. 9C-9D). Whole blood from a healthy donor was incubated with or without EDTA (10 mM) or EGTA (10 mM)±MgCl₂ (10 mM) before incubating with buffer or antigen (PF4; 25 μg/mL+heparin; 0.25 U/mL) and binding of PF4/heparin and C3c to B cells was determined by flow cytometry as described in the materials and methods section.

To examine involvement of the lectin and classical pathways, plasma or whole blood from a healthy donor was pre-incubated with various concentration of monoclonal antibodies to C1q or MBL or murine isotype controls (0-100 μg/mL) before adding PF4/heparin. Complement activation responses to PF4/heparin were assessed by immunoassay (FIG. 9E) or flow cytometry (FIG. 9F-9G). For the flow cytometry experiments, whole blood from a healthy donor was incubated with 100 μg/mL of mouse IgG1 or anti-MBL antibody or anti-C1q antibody before incubating with PF4/heparin. Binding of PF4/heparin and C3c to B cells was determined by flow cytometery as described in the methods section.

Anti-C1q inhibited complement activation by PF4/H in a concentration dependent manner, whereas anti-MBL antibodies or mouse isotype control did not. Additionally, in data not shown, we excluded involvement of individual lectin proteins, ficolin -2 and -3 in complement activation by PF4/heparin complexes. Mass spectrometry data accompanying FIG. 4A did not show correlation of lectin proteins with complement activation phenotype, nor was functional inhibition of ficolin-2 associated with loss of complement activation in our immunoassay (data not shown).

These studies establish that complement is activated by PF4/heparin through the classical complement pathway. Additionally, the studies demonstrate that significant donor variation in circulating IgM levels that can contribute to host susceptibility for immune activation and offer targets for therapeutic intervention to prevent HIT.

### Example 6

### Plasma IgM Co-Localizes with Complement and Antigen on B Cells In Vitro and in Patients Receiving Heparin

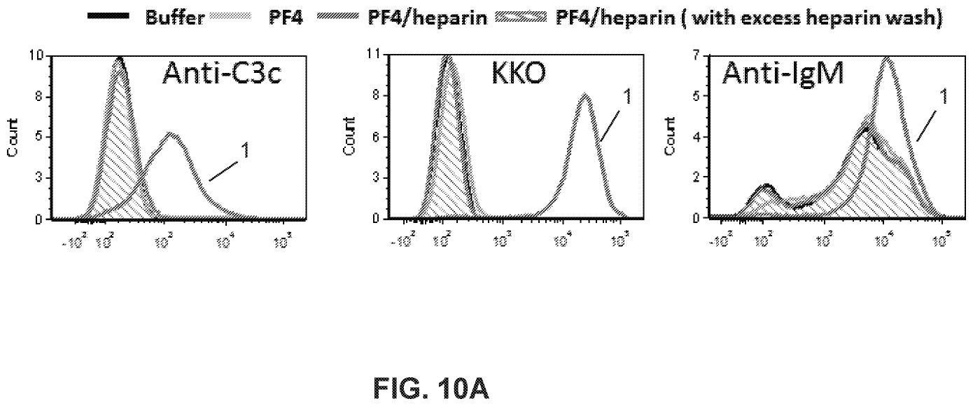

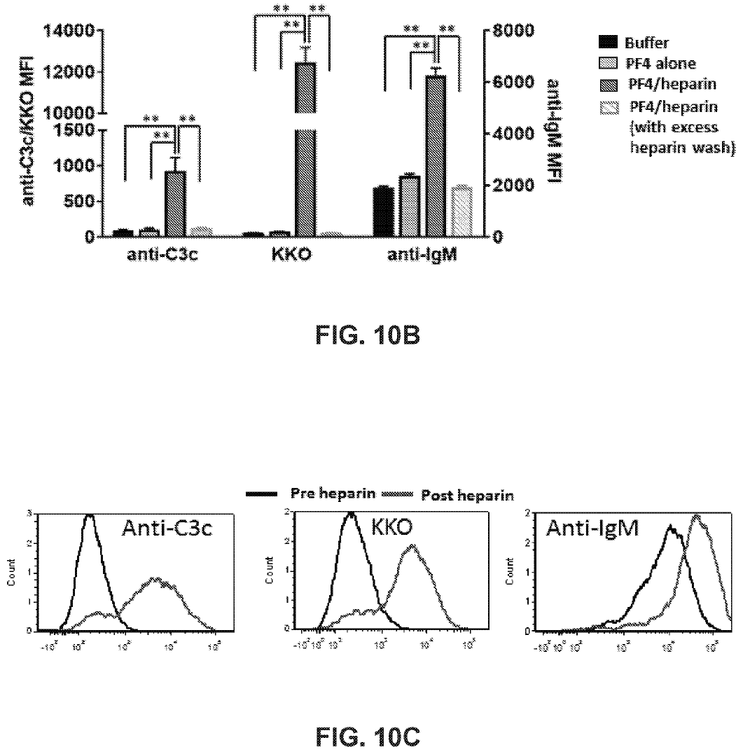

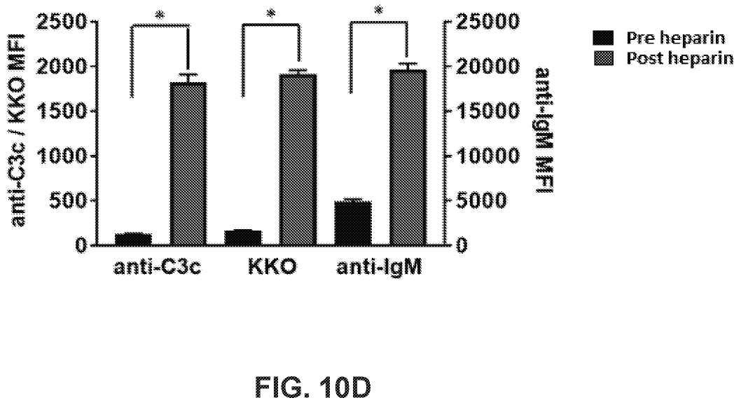

Circulating IgM facilitates antigen transport of particulate antigen through co-localizing with antigen and complement on the surface of non-cognate B cells. To examine if IgM co-localizes with PF4/heparin antigen and complement on B cells, surface IgM on B cells before and after addition of antigen were examined. Incubation of whole blood with buffer or PF4 alone is not associated with antigen or complement deposition on B cells. Under these same conditions, basal levels of IgM were detected on B cells, likely due to binding of anti-IgM antibody to surface BCR. In contrast, when whole blood from a representative healthy donor was incubated with buffer or PF4 (25 μg/mL)±heparin (0.25 U/mL), there was a marked shift in fluorescent signals for complement, PF4/heparin, as well as IgM (FIG. 10A and FIG. 10B). This increase in IgM binding is likely due to plasma-derived antibody (as opposed to surface IgM), as addition of excess heparin reduced PF4/heparin and IgM fluorescence to baseline (FIG. 10A and FIG. 10B).

To explore the clinical relevance of these observations, B cells from patients for co-localization of IgM with PF4/heparin were examined before and after exposure to heparin. Binding of C3c, PF4/H and IgM on B cells in the circulation of heparinized patients was determined by flow cytometry. As shown in FIG. 10C, there was no PF4/heparin, minimal C3c and basal expression of IgM on B cells prior to receiving heparin (black histogram, FIG. 10C and black columns FIG. 10D). By 9 hours after initiating heparin, increased binding of PF4/heparin, complement and IgM was seen on circulating B cells (blue histogram, FIG. 10C and blue columns FIG. 10D). Taken together, these studies show IgM co-localizes with PF4/heparin and complement fragments on circulating B cells and increased bound IgM is likely plasma-derived. Thus, IgM facilitates complement and antigen deposition on B cells in vitro and in patients receiving heparin.

The HIT antigen consists of a complex between cationic PF4 and anionic heparin that generate an array of charged motifs that have similarities to key features of microbial pattern recognition molecules. These properties endow the PF4/heparin ULC with robust complement activating properties.

Herein, the mechanism by which PF4/heparin complexes activate complement was delineated. The results demonstrated wide, but stable, variation in IgM levels in healthy donors that closely correlate with their complement activating responses to PF4/heparin. The data show that polyreactive IgM binds PF4/heparin, triggers activation of the classical complement pathway, and promotes antigen and complement deposition on B cells. Natural IgM mediates this process and complement activation by IgM can be attenuated by classical pathway inhibitors.

The pentameric structure of circulating IgM facilitates high avidity interactions with antigen and allows IgM to have a 1000-fold greater affinity for the classical pathway component C1q compared to IgG. Complement activation is most robust when IgMs (either immune or non-immune) bind to particulate antigens and undergo conformational change to initiate binding of C1q. The data are consistent with these reports, as PF4/heparin complexes by virtue of its charge and size behave as particulate antigen, promote IgM binding and enable complement activation and antigen deposition on B cells (FIGS. 10A and 10B). The observations that circulating B cells from heparinized patients show similar co-localization of IgM, antigen, and complement (FIGS. 10C and 10D) provides not only valuable clinical confirmation of in vitro data, but also validates this IgM-mediated pathway as an important mechanism of immune activation in HIT and suggests plasma IgM levels can provide one biomarker for the risk of seroconversion.

These studies described herein demonstrate that variability in plasma IgM levels correlates with functional complement responses to PF4/heparin. Polyreactive IgM binds PF4/heparin, triggers activation of the classical complement pathway, and promotes antigen and complement deposition on B cells.

The studies described herein also identify IgM and the classical pathway as potential diagnostic and/or therapeutic targets in HIT. In healthy donors, donor phenotypes of complement activation, which correlate with IgM levels (FIGS. 4A and 4C), remain stable over time (FIG. 2C). Additionally, studies shown in FIG. 5C indicate a threshold effect for IgM not seen with PF4/heparin. While additional studies are needed to establish the stability of IgM levels over time in both healthy donors and in patients experiencing infection or inflammation, measurement of IgM at time of heparin exposure may identify patients at low or high risk for sensitization.

Lastly, the results show that the classical pathway can be a therapeutic target in HIT. Disruption of IgM-C1q interactions prevent PF4/heparin mediated complement activation, whereas inhibition of the alternative pathway or MBL activity have no effect (FIG. 9A-9B, 9D). Targeted inhibitors of C1 q/r/s complex such as anti-C1s therapy or broader complement targets such as Cp40, a peptide inhibitor of C3, could be used to prevent HIT seroconversions.

In conclusion, the studies described herein support the following model of complement activation by PF4/heparin complexes. Under physiological conditions, circulating PF4, IgM and C1 do not associate. Once heparin is administered at concentrations that generate PF4/heparin ULCs in the form of particulate antigen, pre-existing IgM binds to PF4/heparin ULCs and undergoes a conformational change (FIG. 1, step A) that initiates binding of C1q followed by activation of the C1 complex. Activation of the classical pathway culminates in activation of the C3 convertase, incorporation of the C3 fragments onto PF4/heparin ULCs (FIG. 1, step B) and subsequent binding of IgM/C3 coated antigen to B cells via complement receptor 2 (CD21) (FIG. 1, step C). Prospective studies in patients receiving heparin therapy will be necessary to define the threshold amounts of IgM and/or PF4/heparin necessary to initiate complement activation and validate the relevance of this mechanism for subsequent HIT antibody formation.

These studies provide new insights into the evolution of the HIT immune response and can provide a biomarker of risk.

Any patents or publications mentioned in this specification are indicative of the levels of those skilled in the art to which the disclosure pertains. These patents and publications are herein incorporated by reference to the same extent as if each individual publication was specifically and individually indicated to be incorporated by reference. In case of conflict, the present specification, including definitions, will control.

One skilled in the art will readily appreciate that the present disclosure is well adapted to carry out the objects and obtain the ends and advantages mentioned, as well as those inherent therein. The present disclosure is presently representative of embodiments, are exemplary, and are not intended as limitations on the scope of the invention. Changes therein and other uses will occur to those skilled in the art which are encompassed within the spirit of the disclosure as defined by the scope of the claims.

1. A method of determining the presence of the risk of developing complement activation by protein/heparin binding complexes in a subject, the method comprising:
(a) obtaining a biological sample from the subject;
(b) determining the presence of plasma IgM in the biological sample; and
(c) if the plasma IgM is determined in an amount greater than a control, administering to the subject a therapeutically effective amount of a compound capable of blocking the classical pathway of complement activation such that the complement activation by protein/heparin complexes is blocked in the subject.

2. The method of claim 1, wherein the protein of the protein/heparin binding complex comprises platelet factor 4 (PF4).

3. The method of claim 1, wherein the compound is a complement inhibitor.

4. The method of claim 3, wherein the complement inhibitor is an antibody, antisense RNA, cDNA, small molecule, fusion protein, peptide, oligonucleotide.

5. The method of claim 3, wherein the complement inhibitor is eculizumab, C1-INH, anti-C1q antibodies, anti-C1s antibodies, compstatin, or anti-CD21 inhibitors.

6. The method of claim 1, wherein a concentration of plasma IgM in the biological sample at about 200 μg/mL to about 3000 μg/mL indicates an increased likelihood of developing complement activation by protein/heparin binding complexes in the subject.

7. The method of claim 1, wherein the compound is administered in a pharmaceutically acceptable composition.

8. The method of claim 7, wherein the pharmaceutically acceptable composition comprises a pharmaceutically acceptable carrier.

9. (canceled)

10. The method of claim 1, wherein the compound is administered intravenously, intraperitonealy, intramuscularly, subcutaneously, or transdermaly.

11. The method of claim 1, wherein the biological sample is tissues, cells, biopsies, blood, lymph, serum, plasma, urine, saliva, mucus, or tears.

12. (canceled)

13. A method of determining the presence of complement activation by protein/heparin binding complexes in a subject, the method comprising:
(a) obtaining a biological sample from the subject;
(b) determining the presence of plasma IgM in the biological sample;
(c) if the plasma IgM is determined in an amount greater than the control, administering to the subject a therapeutically effective amount of a compound capable of blocking the classical pathway of complement activation such that complement activation by protein/heparin complexes is blocked in the subject.

14. The method of claim 13, wherein the protein of the protein/heparin binding complex comprises platelet factor 4 (PF4).

15. The method of claim 13, wherein the compound is a complement inhibitor.

16. The method of claim 13, wherein the complement inhibitor is an antibody, antisense RNA, cDNA, small molecule, fusion protein, peptide, oligonucleotide.

17. The method of claim 13, wherein the complement inhibitor is eculizumab, C1-INH, anti-C1q antibodies, anti-C1s antibodies, compstatin, or anti-CD21 inhibitors.

18. The method of claim 13, wherein the amount of plasma IgM in the biological sample is about 200 μg/mL to about 3000 μg/mL.

19. The method of claim 13, wherein the compound is administered in a pharmaceutically acceptable composition.

20. The method of claim 19, wherein the pharmaceutically acceptable composition comprises a pharmaceutically acceptable carrier.

21. (canceled)

22. The method of claim 13, wherein the compound is administered intravenously, intraperitonealy, intramuscularly, subcutaneously, or transdermaly.

23. The method of claim 13, wherein the biological sample is tissues, cells, biopsies, blood, lymph, serum, plasma, urine, saliva, mucus, or tears.

24.-40. (canceled)
